(19) United States
(12) Patent Application Publication
(10) Pub. No.: US 2024/0011084 A1
(43) Pub. Date: Jan. 11, 2024

(54) METHODS FOR FULL-LENGTH REVERSE TRANSCRIPTION PCR OF LONG RNAs CONTAINING MODIFIED RIBONUCLEOSIDES USING A THERMOSTABLE REVERSE TRANSCRIPTASE

(71) Applicant: Northwestern University, Evanston, IL (US)

(72) Inventors: Michael C. Jewett, Evanston, IL (US); Michael J. Hammerling, Evanston, IL (US)

(21) Appl. No.: 18/251,596

(22) PCT Filed: Nov. 2, 2021
(86) PCT No.: PCT/US21/72178

§ 371 Date: May 3, 2023

Related U.S. Application Data
(60) Provisional application No. 63/109,156, filed on Nov. 3, 2020.

(51) Int. Cl. C12Q 1/6858; C12Q 1/6806

(52) U.S. Cl. CPC C12Q 1/6858; C12Q 1/6806; C12Q 2600/154

(57) ABSTRACT

Disclosed are methods, components, compositions, and kits for preparing DNA molecules by reverse transcribing RNA templates that comprise modified ribonucleosides. The disclosed methods, components, compositions, and kits utilize or comprise thermostable enzymes having RNA-dependent DNA polymerase activity, otherwise referred to as reverse transcriptases.

### CROSS-REFERENCE TO RELATED APPLICATIONS

This application claims the benefit of U.S. Application No. 63/109,156 filed Nov. 3, 2020, the content of which is incorporated herein by reference in its entirety.

### STATEMENT REGARDING FEDERALLY SPONSORED RESEARCH OR DEVELOPMENT

This invention was made with government support under grant numbers W911NF-18-1-0181 and W911NF-16-1-0372 awarded by the Army Research Office, Department of Defense. The government has certain rights in the invention.

### SEQUENCE LISTING

A Sequence Listing accompanies this application and is submitted as an ASCII text file of the sequence listing named “702581_02047_ST25.txt” which is 9,237 bytes in size and was created on Nov. 2, 2021. The sequence listing is electronically submitted via EFS-Web with the application and is incorporated herein by reference in its entirety.

### BACKGROUND

The present invention generally relates to methods for in vitro synthesis of polynucleotides. More specifically, the present invention relates to methods for DNA molecules by reverse transcribing RNA templates that comprise modified ribonucleosides using thermostable reverse transcriptases.

The conversion of RNA from living cells or in vitro reactions into DNA, a process called reverse-transcription polymerase chain reaction (RT-PCR), is essential for the purposes of technologies such as directed evolution and for the study of RNA function in cells. However, RT-PCR of long RNA can be challenging, especially if the RNA contains posttranscriptional modifications.

### SUMMARY

Disclosed are methods, components, compositions, and kits for preparing DNA molecules by reverse transcribing RNA templates that comprise modified ribonucleosides. The disclosed methods, components, compositions, and kits may utilize or comprise thermostable enzymes having RNA-dependent DNA polymerase activity, otherwise referred to as thermostable reverse transcriptases.

In one aspect of the current disclosure, methods for preparing a DNA molecule from an RNA template via reverse transcription, wherein the RNA template comprises one or more modified ribonucleosides are provided. In some embodiments, the methods comprise reacting a reaction mixture comprising: (i) a thermostable enzyme that comprises RNA-dependent DNA polymerase activity, (ii) the RNA template, (iii) one or more oligonucleotide primers that hybridize to the RNA template, and (iv) reagents for performing reverse transcription of the RNA template. In some embodiments, the one or more oligonucleotide primers hybridize to a region of the RNA template spanning the one or more modified ribonucleosides. In some embodiments, the reaction mixture is reacted for at least about 14 minutes or longer. In some embodiments, the reaction mixture is reacted for no more than about 6 minutes or less. In some embodiments, the reaction mixture is reacted at a temperature greater than about 50° C., 55° C., 60° C., 65° C., 70° C., 75° C., 80° C., 85° C., 90° C., 95° C., or higher to prepare the DNA molecule from the RNA template. In some embodiments, the thermostable enzyme additionally comprises DNA-dependent DNA polymerase activity. In some embodiments, the enzyme is RTX. In some embodiments, the reaction mixture comprises an additional different enzyme that comprises DNA-dependent DNA polymerase activity. In some embodiments, the reaction mixture further comprises: (iv) a forward primer and a reverse primer that hybridize to the DNA molecule, and (v) reagents for amplifying the prepared DNA molecule, and the method further comprises amplifying the prepared DNA molecule via performing a polymerase chain reaction (PCR) amplification. In some embodiments, the method comprises performing an elongation step in which reverse transcription of the RNA template occurs and an amplification step during which amplification of the prepared DNA occurs. In some embodiments, the RNA template is at least about 200, 400, 600, 800, 1000, 1200, 1400, 1600, 1800, 2000, 2200, 2400, 2600, 2800, 3000 nucleotides in length or longer, and the method prepares a DNA molecule corresponding to the full-length RNA template. In some embodiments, the RNA template comprises ribosomal RNA. In some embodiments, the RNA template comprises prokaryotic rRNA. In some embodiments, the RNA template comprises eukaryotic rRNA. In some embodiments, the RNA template comprises E. coli 16s rRNA or E. coli 23s rRNA. In some embodiments, the modified ribonucleoside is a naturally occurring modified ribonucleoside. In some embodiments, the modified ribonucleoside comprises a methylated base. In some embodiments, the modified ribonucleoside comprises a methylated ribose. In some embodiments, the one or more modified ribonucleosides are selected from pseudouridine, N⁷-methylguanosine, N²-methylguanosine, N⁴-methylcytosine, N⁴,2′-O-methylcytosine, 5-methylcytosine, 5-hydroxymethylcytosine, N³-methyluridine, N⁶,N⁶-dimethyladenosine, N¹-methylguanosine, 5-methyluridine, N⁶-methyladenosine, N³-methylpseudouridine, 5-methyluridine, 2′-O-methylguanosine, dihydrouridine, 2′-O-methylcytosine, N²-methyladenosine, and 2′-O-methyluridine. In some embodiments, the one or more modified ribonucleosides are selected from N²-methylguanosine, N⁴,2′-O-methylcytosine, N³-methyluridine, N⁶,N⁶-dimethyladenosine, N¹-methylguanosine, N⁶-methyladenosine, N³-methylpseudouridine, 2′-O-methylguanosine, dihydrouridine, 2′-O-methylcytosine, N²-methyladenosine, and 2′-O-methyluridine. In some embodiments, the one or more modified ribonucleosides are selected from N³-methyluridine, N⁶,N⁶-dimethyladenosine, N¹-methylguanosine, and N³-methylpseudouridine.

In another aspect of the current disclosure methods for identifying a modified ribonucleoside at a position in an RNA template are provided. In some embodiments, the method comprises: (a) preparing a DNA molecule from the RNA template via reverse transcription by reacting a reaction mixture comprising: (i) a thermostable enzyme that comprises RNA-dependent DNA polymerase activity, (ii) the RNA template, (iii) one or more oligonucleotide primers that hybridize to the RNA template, and (iv) reagents for performing reverse transcription of the RNA template comprising deoxyribonucleotides which optionally are labeled; and (b) identifying the incorporated deoxyribonucleotides in the DNA molecule, (c) generating a mutation spectrum based on the incorporated deoxyribonucleotides at a position in the DNA molecule, (d) comparing the generated mutation spectrum to a reference mutation spectrum which is characteristic of the modified ribonucleoside, and (e) identifying the modified ribonucleoside in the RNA template at a position corresponding to the position in the DNA molecule. In some embodiments, the modified ribonucleoside is selected from pseudouridine, N⁷-methylguanosine, N²-methylguanosine, N⁴-methylcytosine, N⁴,2′-O-methylcytosine, 5-methylcytosine, 5-hydroxymethylcytosine, N³-methyluridine, N⁶,N⁶-dimethyladenosine, N¹-methylguanosine, 5-methyluridine, N⁶-methyladenosine, N³-methylpseudouridine, 5-methyluridine, 2′-O-methylguanosine, dihydrouridine, 2′-O-methylcytosine, N²-methyladenosine, and 2′-O-methyluridine. In some embodiments, the modified ribonucleoside is selected from N²-methylguanosine, N⁴,2′-O-methylcytosine, N³-methyluridine, N⁶,N⁶-dimethyladenosine, N¹-methylguanosine, N⁶-methyladenosine, N³-methylpseudouridine, 2′-O-methylguanosine, dihydrouridine, 2′-O-methylcytosine, N²-methyladenosine, and 2′-O-methyluridine. In some embodiments, the modified ribonucleoside is selected from N³-methyluridine, N⁶,N⁶-dimethyladenosine, N¹-methylguanosine, and N³-methylpseudouridine.

In another aspect of the current disclosure, methods for preparing a DNA molecule from an RNA template via reverse transcription, wherein the RNA template comprises one or more modified ribonucleosides are provided. In some embodiments, the method comprising reacting a reaction mixture comprising: (i) a thermostable enzyme that comprises RNA-dependent DNA polymerase activity, (ii) the RNA template, (iii) one or more oligonucleotide primers that hybridize to the RNA template and comprise at least one bridging oligonucleotide primer that hybridizes to a region of the RNA template spanning the one or more modified ribonucleosides, and (iv) reagents for performing reverse transcription of the RNA template. In some embodiments, the modified ribonucleoside is selected from pseudouridine, N⁷-methylguanosine, N²-methylguanosine, N⁴-methylcytosine, N⁴,2′-O-methylcytosine, 5-methylcytosine, 5-hydroxymethylcytosine, N³-methyluridine, N⁶,N⁶-dimethyladenosine, N¹-methylguanosine, 5-methyluridine, N⁶-methyladenosine, N³-methylpseudouridine, 5-methyluridine, 2′-O-methylguanosine, dihydrouridine, 2′-O-methylcytosine, N²-methyladenosine, and 2′-O-methyluridine. In some embodiments, the modified ribonucleoside is selected from N²-methylguanosine, N⁴,2′-O-methylcytosine, N³-methyluridine, N⁶,N⁶-dimethyladenosine, N¹-methylguanosine, N⁶-methyladenosine, N³-methylpseudouridine, 2′-O-methylguanosine, dihydrouridine, 2′-O-methylcytosine, N²-methyladenosine, and 2′-O-methyluridine. In some embodiments, the modified ribonucleoside is selected from N³-methyluridine, N⁶,N⁶-dimethyladenosine, N¹-methylguanosine, and N³-methylpseudouridine. In some embodiments, the bridging oligonucleotide primer is at least about 10, 15, 20, 25, 30, 35, 45, or 50 nucleotides in length and spans the modified ribonucleoside by at least about 10, 15, 20, or 25 nucleotides 5′ to the position of the modified ribonucleoside and/or spans the modified ribonucleoside by at least about 5, 10, 15, 20, or 25 nucleotides 3′ to the position of the modified ribonucleoside. In some embodiments, the enzyme is RTX.

### BRIEF DESCRIPTION OF THE DRAWINGS

FIG. 1A-B: Locations and structures of post-transcriptional modifications of the E. coli rRNAs. (A) Diagram of the established methods for converting rRNA into rDNA and the methods developed in this work. rRNA from ribosomes selected by directed evolution is purified and must be reverse transcribed to recover successful genotypes. In prior work, rDNA libraries have focused on small portions of the ribosome which lack post-transcriptional modifications that block reverse transcription to enable library recovery (top arrows) This work documents the development of methods to reverse transcribe the entirety of the 16S and 23S rRNAs to enable construction and selection of libraries covering the whole ribosome (bottom arrows). (B) The E. coli rRNAs are extensively modified, with the 16S and 23S rRNAs containing as many as 11 and 24 modifications, respectively. In addition to their significance in ribosome assembly and function, these modifications may be categorized by the impact on RT-PCR. Permissive modifications (cyan) have little to no impact on polymerase extension, pausing modifications (orange) cause the polymerase to pause briefly, and blocking modifications (red) cause the polymerase to stop and fall off the message. Blocking modifications generally sterically disrupt the Watson-Crick interface, while pausing modifications may modestly interfere with Watson-Crick base-pairing or polymerase binding to the RNA backbone.

FIG. 2A-C: Post-transcriptional modifications inhibit RT-PCR of the rRNA. (A) RT-PCR spanning positions 725 to 1858 of MRE600 23S rRNA from cells yields a robust product, while PCR using DNA polymerases PfX, Phusion (Phn) or Q5 do not produce the correct product. (B) Diagram of the RT-PCR products attempted using RTX. Products were chosen to yield information about the ability of RTX to traverse PTxMs. (C) For the 23S rRNA, products spanning post-transcriptional modifications do not produce product in 4 of 6 cases, (products 2, 7, 8, and 9) suggesting that these modifications inhibit RTX polymerization. However, the 2 products containing PTxMs which did appear to produce the correct product (3 and 4, shaded cells) suggest that RTX can traverse blocking PTxMs if given enough time. For the 16S rRNA, all products except 14 conclusively form a product of the correct length, while product 14 appears to form a minor product of approximately correct size. Gels representative of three independent experiments.

FIG. 3A-C: Bridging primers enable RT-PCR of the entire 23S rRNA. (A) Diagram of the bridging primer sets tested for the 23S rRNA at top. An agarose gel of RT-PCRs attempted on the 23S rRNA using RTX and combinations of bridging primers is depicted. The sets of bridging primers used in each lane are indicated in each column of the table below the gel, with “+” indicating the presence and “−” indicating the absence of the primer set. Shaded columns in the table indicate primer combinations which produced primarily the correct full-length product. The minimal primers for successful RT-PCR was primer sets 1 and 2 together, which span blocking PTxMs. (B) RT-PCR of the full-length 23S rRNA using RTX and primer sets 1 and 2 together from a gradient of rRNA concentrations. (C) RT-PCR of >97% of the 16S rRNA (1497 bases) using a single set of bridging primers. In all panels, the asterisk represents blocking PTxMs. Gels representative of three independent experiments.

FIG. 4A-D: RT-PCR of entire 23S rRNA using long elongation steps. (A) Diagram of the RT-PCR reactions of the 23S rRNA attempted. Products are labeled with letters from A-P from the top down. (B) RT-PCR elongation steps of 8, 10, 12, and 14 minutes were attempted (agarose gel of 14 minute reactions shown). Longer elongation steps promote formation of correct products that were not obtained using standard reaction conditions. (C) RT-PCR elongation steps of 20, 25, and 30 minutes were performed (agarose gel of 25 minute reactions shown). Elongation steps of this length enable production of lengths up to but not including the full-length product. (D) The reverse primer was varied in the full-length 23S rRNA RT-PCR reaction to troubleshoot this reaction. Correct full-length product was obtained in all reactions with a new reverse primer, though with some smaller off-target products as well. Arrow indicates correct full-length RT-PCR product of the 23S rRNA. Gels representative of three independent experiments.

FIG. 5A-B: Two-enzyme strategy for RT-PCR of full-length 23S and 16S rRNA. (A) Amplification of 23S cDNA from purified rRNA was attempted using RTX alone or RTX reactions spiked into Q5 polymerase PCRs after 1, 3, or 5 long (30 minute) RTX extension steps. Reactions were performed on two-fold serially diluted rRNA ranging from 50 ng to 40 pg total rRNA. Little difference was noted between the RTX alone and RTX+Q5 conditions for the 23S rRNA, indicating that a two-enzyme strategy does not improve recovery of 23S rDNA. (B) The same conditions were tested for recovery of 16S cDNA. The two-enzyme strategy does improve reliability of the recovery of 16S cDNA. Gels representative of three independent experiments.

FIG. 6A-B: Error rate of RTX traversing posttranscriptional modifications. (A) The average error rate (n=4 independent RT-PCR reactions) of each position of the 23s rRNA is plotted and color-coded for positions with no PTxM (black), or polymerization-permissive (blue), -pausing (yellow) or -blocking (red) PTxM. The inset is a zoomed in plot of positions 1600-2700 which contain the bulk of the permissive and pausing PTxMs to enable greater resolution of plotting. Blocking PTxMs induce a greatly elevated error rate, while select positions with pausing PTxMs (1618, 2552) also show an increase in error rate compared to the trend for that region of the 23S rRNA. For most points, standard deviation error bars are smaller than the diameter of the point and thus not depicted for clarity of plotting. (B) Pie charts depicting the RTX mutation spectrum at positions 745 and 1915 with blocking PTxMs m¹G and m³Ψ, respectively.

FIG. 7: RT-PCR of the 23S rRNA using bridging primer sets 1 and 2. Inclusion of the minimal necessary set of bridging primers enables full-length RT-PCR of the 23S rRNA with minimal off products. While 3 minute elongation steps are sufficient to produce the correct product given high template concentration, 6 minute elongation enables correct product formation even at the lowest concentration of template. Template rRNA from 50 ng to 40 pg. Gels representative of three independent experiments.

FIG. 8: RT-PCR of the 16S rRNA using bridging primers. Inclusion of bridging primers spanning m²G and m⁵C at positions 966-967 enables full-length RT-PCR of the 16S rRNA. The shortest tested elongation step of 2 minutes was sufficient to produce the correct product down to the lowest concentration tested. Gels representative of three independent experiments.

FIG. 9: RT-PCR of 23S rRNA using long extension steps. Diagram depicts approximate lengths of products A through P of the 23S rRNA. Longer elongation durations enables RT-PCR of larger portions of the 23S rRNA. In particular, products K, L, and O are absent in reactions with 8 minute elongation steps, but present in reactions with 14 minute extension steps. Primers used in these reactions are listed in Supplementary Table 3. Gels representative of three independent experiments.

FIG. 10: Further optimization of elongation step length for RT-PCR of the 23S rRNA. The fragments that were intransigent to RT-PCR in Supplementary FIG. 2 were subjected to even longer elongation steps between 20-30 minutes. With 30 minute extension steps, all correct products are formed except the full-length product P. Gels representative of three independent experiments.

FIG. 11: Full-length 23S RT-PCR with different reverse primers. Success of RT-PCR of all products except product P lead to the hypothesis that the reverse primer in this reaction was responsible for failure to generate full-length product. Five alternative reverse primers were implemented in products P-2 through P-6, all of which generated correct full-length product. Sequences of reverse primers used in these reactions can be found in Supplementary Table 4. Gels representative of three independent experiments.

FIG. 12: Varying short cycle elongation duration confirms necessity of a longer elongation step. Three cycles using thirty-minute elongation steps were performed in each set of reactions followed by 35 cycles using elongation steps of the specified duration. Both 5 and 6 minute reaction sets efficiently produce full-length 23S cDNA. Gels representative of three independent experiments.

FIG. 13: RT-PCR of 16S rRNA without bridging primers produces correct product plus aberrant off-products. The 16S rRNA is able to be reverse transcribed using a single long extension step and no bridging primers. While the correct product is produced, smaller and larger off-products are also produced. Gels representative of three independent experiments.

### DETAILED DESCRIPTION

The technology disclosed herein addresses the need for methods to enable RT-PCR of very long RNAs and also provides a means by which to identify the post-transcriptional modifications which are present in RNAs expressed in living cells. The technology disclosed herein can be used to catalog the modifications of RNAs in living cells from any organism, and to begin the study of their function.

In some methods exemplified herein, the inventors utilize a recently developed, synthetic, thermostable reverse transcriptase to perform RT-PCR on long RNA templates which contain modified ribonucleosides. These modifications may normally block or inhibit RT-PCR of RNA templates comprising these modified ribonucleosides. Here, the inventors describe two methods for overcoming this block and inhibition of RT-PCR or RNA templates comprising these modified ribonucleosides. The first method uses bridging primers to circumvent the need for the reverse transcriptase to reverse-transcribe through the modified ribonucleosides. The second method uses relatively long time period for extension steps, which allow the reverse transcriptase to read through the modified ribonucleosides. This second method also results in a characteristic mutation spectrum for the modified ribonucleosides which is read through by the reverse transcriptase. This spectrum can be used to identify modified ribonucleosides in naturally occurring RNAs such as long non-coding RNAs.

### Definitions and Terminology

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “an oligonucleotide primer” should be interpreted to mean “one or more oligonucleotide primers” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into subranges as discussed above.

A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Polynucleotides and Synthesis Methods

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

Oligonucleotides and polynucleotides may optionally include one or more non-standard nucleoside(s), nucleoside analog(s) and/or modified nucleosides which may be naturally or non-naturally occurring and may include modified ribonucleosides and modified deoxyribonucleosides. Examples of naturally occurring modified nucleosides include, but are not limited to pseudouridine, N⁷-methylguanosine, N²-methylguanosine, N⁴-methylcytosine, N⁴,2′-O-methylcytosine, 5-methylcytosine, 5-hydroxymethylcytosine, N³-methyluridine, N⁶,N⁶-dimethyladenosine, N¹-methylguanosine, 5-methyluridine, N⁶-methyladenosine, N³-methylpseudouridine, 5-methyluridine, 2′-O-methylguanosine, dihydrouridine, 2′-O-methylcytosine, N²-methyladenosine. Other modified nucleosides may include, but are not limited to, and 2′-O-methyluridine, diaminopurine, S 2 T, 5-fluorouracil, 5-bromouracil, 5-chlorouracil, 5-iodouracil, hypoxanthine, xantine, 4-acetylcytosine, 5-(carboxyhydroxylmethyl)uracil, 5-carb oxy methylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-adenine, 7-methylguanine, 5-methylaminomethyluracil, 5-methoxy aminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), at the sugar moiety (e.g., via methylation at a hydroxyl such as 2′-O-methyl), or at the phosphate backbone.

The term “reverse transcription reaction” refers to any chemical reaction, including an enzymatic reaction, which results in synthesis of a DNA molecule from an RNA template, where the DNA molecule exhibits reverse complementarity to the RNA template molecule. Enzymes useful for performing a reverse transcription reaction include thermostable enzymes having reverse transcriptase activity, for example enzymes that are thermostable at temperatures greater than about 50° C., 55° C., 60° C., 65° C., 70° C., ° C., 80° C., 85° C., 90° C., 95° C. or higher. As used herein, “reverse transcription” refers to the function of an enzyme with RNA dependent DNA polymerase activity. In some embodiments, reverse transcription is performed by a “reverse transcriptase”. In some embodiments, the reverse transcriptase is RTX.

As used herein, “RNA template” refers to an RNA polynucleotide that is used as a template for a reaction including, for example, a reverse transcription reaction.

The term “amplification reaction” refers to any chemical reaction, including an enzymatic reaction, which results in increased copies of a template nucleic acid sequence or results in transcription of a template nucleic acid. Amplification reactions include the polymerase chain reaction (PCR), including Real Time PCR (see U.S. Pat. Nos. 4,683,195 and 4,683,202; PCR Protocols: A Guide to Methods and Applications (Innis et al., eds, 1990)), the ligase chain reaction (LCR) (see Barany et al., U.S. Pat. No. 5,494,810), and reverse transcriptase (PCR) (RT-PCR). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

The terms “target,” “target sequence”, “target region”, and “target nucleic acid,” as used herein, are synonymous and refer to a region or sequence of a nucleic acid which is to be amplified, sequenced, or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26 (3/4):227-259; and Owczarzy et al., 2008, Biochemistry, 47: 5336-5353, which are incorporated herein by reference).

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6, 8, 10, 12, 14, 16, 18, 20, 25, 30, 35, 40, 45, 50, 55, 60, 65, 70, 75, 80, 85, 90 to about 100 nucleotides, including intermediate ranges, such as from 15 to 35 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

The term “promoter” refers to a cis-acting DNA sequence that directs RNA polymerase and other trans-acting transcription factors to initiate RNA transcription from the DNA template that includes the cis-acting DNA sequence.

As used herein, the term “sequence defined biopolymer” refers to a biopolymer having a specific primary sequence. A sequence defined biopolymer can be equivalent to a genetically-encoded defined biopolymer in cases where a gene encodes the biopolymer having a specific primary sequence.

As used herein, “expression template” refers to a nucleic acid that serves as substrate for transcribing at least one RNA that can be translated into a sequence defined biopolymer (e.g., a polypeptide or protein). Expression templates include nucleic acids composed of DNA or RNA. Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

The term “reaction mixture,” as used herein, refers to a solution containing reagents necessary to carry out a given reaction. A reaction mixture is referred to as complete if it contains all reagents necessary to perform the reaction. Components for a reaction mixture may be stored separately in separate container, each containing one or more of the total components. Components may be packaged separately for commercialization and useful commercial kits may contain one or more of the reaction components for a reaction mixture. In some embodiments, a reaction mixture comprises a thermostable enzyme that comprises RNA-dependent DNA polymerase activity, the RNA template, one or more oligonucleotide primers that hybridize to the RNA template, and reagents for performing reverse transcription of the RNA template. In some embodiments, the reaction mixture further comprises: a forward primer and a reverse primer that hybridize to the DNA molecule, and reagents for amplifying the prepared DNA molecule, and the method further comprises amplifying the prepared DNA molecule via performing a polymerase chain reaction (PCR) amplification.

As used herein, “thermostable” refers to the property of an enzyme to be resistant to heat-mediated loss of function. In some embodiments, thermostable enzymes comprise RTX reverse transcriptase.

The steps of the methods described herein can be performed in any suitable order unless otherwise indicated herein or otherwise clearly contradicted by context. The steps may be repeated or reiterated any number of times to achieve a desired goal unless otherwise indicated herein or otherwise clearly contradicted by context.

Methods for Full-Length Reverse Transcription PCR of Long RNAs Containing Modified Ribonucleosides Using a Thermostable Reverse Transcriptase

The subject matter disclosed herein relates to methods, components, compositions, and kits for preparing DNA molecules by reverse transcribing RNA templates that comprise modified ribonucleosides. The modified ribonucleosides may inhibit or block reverse transcription using standard non-thermostable reverse transcriptases under standard conditions. The disclosed methods, components, compositions, and kits utilize or comprise thermostable enzymes having RNA-dependent DNA polymerase activity, otherwise referred to as thermostable reverse transcriptases, at relatively high temperatures in order to prepare DNA molecules by reverse transcribing RNA templates that comprise modified ribonucleosides.

The disclosed methods typically include a step of preparing a DNA molecule from an RNA template via reverse transcription (RT). The RNA template typically includes one or more naturally or non-naturally modified ribonucleosides. The disclosed methods may utilize a reaction mixture where in the methods the reaction mixture is reacted in order to prepare the DNA molecule. As such, the reaction mixture may comprise one or more of: (i) a thermostable enzyme that comprises RNA-dependent DNA polymerase activity, (ii) the RNA template, wherein the RNA template comprises the one or more modified ribonucleosides, (iii) one or more oligonucleotide primers that hybridize to the RNA template, and (iv) reagents for performing reverse transcription of the RNA template.

The reverse transcription step of the disclosed methods may be performed at a relatively high temperature. In some embodiments, the reaction mixture is reacted at a temperature greater than about 50° C., 55° C., 60° C., 65° C., 70° C., 75° C., 80° C., 85° C., 90° C., 95° C., or higher to prepare the DNA molecule from the RNA template via reverse transcription.

The disclosed methods typically include a step wherein reverse transcription is performed and/or elongation of an oligonucleotide primer is performed. In some embodiments, reverse transcription and/or elongation is performed at a temperature greater than about 50° C., 55° C., 60° C., 65° C., 70° C., 75° C., 80° C., 85° C., 90° C., 95° C.

In the disclosed methods, the reaction mixture may be reacted for a suitable time period. In some embodiments, the reaction mixture is reacted for at least about 2, 4, 6, 8, 10, 12, 14, 16, 18, 20, 25, 30 minutes or longer. In other embodiments, the reaction mixture is reacted for no more than about 30, 25, 20, 18, 16, 14, 12, 10, 8, 6, 4, 2 minutes or less.

The disclosed methods typically include a step wherein reverse transcription is performed and/or elongation of an oligonucleotide primer is performed for a suitable time period. In some embodiments, reverse transcription and/or elongation is performed for at least about 2, 4, 6, 8, 10, 12, 14, 16, 18, 20, 25, 30 minutes or longer. In other embodiments, reverse transcription and/or elongation is performed for no more than about 30, 25, 20, 18, 16, 14, 12, 10, 8, 6, 4, 2 minutes or less.

The disclosed methods may utilize relatively long RNA templates. In some embodiments, the RNA templates utilized in the disclosed methods have a length which is at least about 200, 400, 600, 800, 1000, 1200, 1400, 1600, 1800, 2000, 2200, 2400, 2600, 2800, 3000 nucleotides. Preferably, the disclosed methods may be performed in order to prepare a DNA molecule corresponding to the full-length RNA template, i.e., a prepared DNA molecule corresponding to an RNA template having a length which is at least about 200, 400, 600, 800, 1000, 1200, 1400, 1600, 1800, 2000, 2200, 2400, 2600, 2800, 3000 nucleotides.

The disclosed methods typically utilize a thermostable enzyme that comprises RNA-dependent DNA polymerase activity, where the thermostable enzyme is present in the reaction mixture of the disclosed methods. In some embodiments, the thermostable enzyme additionally comprises DNA-dependent DNA polymerase activity. Accordingly, the thermostable enzyme may utilize both of RNA templates and DNA templates to prepare DNA.

The disclosed methods typically utilize a thermostable enzyme that comprises RNA-dependent DNA polymerase activity. In some embodiments, the disclosed methods may utilize a different enzyme that comprises DNA-dependent DNA polymerase activity. In some embodiments, the reaction mixture of the disclosed methods comprises the thermostable enzyme that comprises the RNA-dependent DNA polymerase activity, and the reaction mixture further comprises an additional different enzyme that comprises the DNA-dependent DNA polymerase activity.

The disclosed methods typically utilize an oligonucleotide primer for performing reverse transcription and/or elongation of a template (e.g., an RNA template and/or a DNA template). As such, the reaction mixture of the disclosed methods typically includes a one or more oligonucleotide primers that hybridize to a template (e.g., an RNA template and/or a DNA template). For example, the reaction mixture may comprise a reverse oligonucleotide primer that is utilized to reverse transcribe an RNA template and prepare a DNA molecule. In some embodiments, the reaction mixture comprises: a forward oligonucleotide primer and a reverse oligonucleotide primer that hybridize to the DNA molecule (i.e., an oligonucleotide primer pair), which may be utilized to amplify the prepared DNA molecule.

The disclosed methods typically include a step of reverse transcribing an RNA template to prepare a DNA molecule. As such, the reaction mixture of the disclosed methods typically includes reagents for performing reverse transcription of the RNA template (e.g., dNTP's, a buffer, divalent cations, salts, crowding agents, and the like). The disclosed methods further may include an additional step of amplifying the prepared DNA molecule. As such, the reaction mixture of the disclosed methods may include reagents for performing amplification of the prepared DNA molecule (e.g., dNTP's, a buffer, divalent cations, salts, crowding agents, and the like). Accordingly, the disclosed methods may include performing an elongation step in which reverse transcription of the RNA template occurs and performing an amplification step during which amplification of the prepared DNA occurs.

The reaction mixture for performing reverse transcription of the RNA template may be the same or different than the reaction mixture for performing amplification of the prepared DNA molecule. In the disclosed methods, the reverse transcription step of the disclosed method and the amplification step of the disclosed methods may be performed in the same reaction vessel or in separate reaction vessels.

The disclosed methods typically utilize an RNA template that comprises one or more modified ribonucleosides. In some embodiments, the RNA template of the disclosed methods comprises ribosomal RNA (rRNA), which may include prokaryotic rRNA and eukaryotic rRNA. Suitable rRNA for the disclosed methods may include, but is not limited to E. coli 16s rRNA or E. coli 23s rRNA.

The RNA template of the disclosed methods typically includes one or more modified ribonucleosides. In some embodiments, the modified ribonucleosides is a naturally occurring modified ribonucleosides. Modified ribonucleosides may include, but are not limited to nucleosides comprising a methylated base and/or a methylated ribose.

In some embodiments, the RNA template of the disclosed methods includes one or more modified ribonucleosides selected from pseudouridine, N⁷-methylguanosine, N²-methylguanosine, N⁴-methylcytosine, N⁴,2′-O-methylcytosine, 5-methylcytosine, 5-hydroxymethylcytosine, N³-methyluridine, N⁶,N⁶-dimethyladenosine, N¹-methylguanosine, 5-methyluridine, N⁶-methyladenosine, N³-methylpseudouridine, 5-methyluridine, 2′-O-methylguanosine, dihydrouridine, 2′-O-methylcytosine, N²-methyladenosine, and 2′-O-methyluridine. In particular, the RNA template of the disclosed methods includes one or more modified ribonucleosides selected from N²-methylguanosine, N⁴,2′-O-methylcytosine, N³-methyluridine, N⁶,N⁶-dimethyladenosine, N¹-methylguanosine, N⁶-methyladenosine, N³-methylpseudouridine, 2′-O-methylguanosine, dihydrouridine, 2′-O-methylcytosine, N²-methyladenosine, and 2′-O-methyluridine. More particularly, the RNA template of the disclosed methods includes one or more modified ribonucleosides selected from N³-methyluridine, N⁶,N⁶-dimethyladenosine, N¹-methylguanosine, and N³-methylpseudouridine.

The disclosed methods typically utilize one or more primer that hybridize to the RNA template comprising the one or more modified ribonucleosides. In some embodiments of the disclosed methods, the disclosed methods may utilize oligonucleotide primers which span or bridge a region of the RNA template that comprises the one or more modified ribonucleosides.

Applications of the disclosed technology may include, but are not limited to: (i) directed evolution of RNAs (see, e.g., U.S. Published Application No. 20170306320, the content of which is incorporated herein by reference in its entirety); (ii) directed evolution of large proteins with long coding RNAs; (iii) study of RNA modification in living cells; (iv) RNA sequencing; and (v) ribosome engineering (see, e.g., U.S. Published Application No. 20170073381 and U.S. Published Application No. 20160060301, the contents of which are incorporated herein by reference in their entireties).

Advantages of the disclosed technology may include, but are not limited to: (i) the disclosed technology enables single enzyme RT-PCR of relatively long RNA templates; (ii) the disclosed technology enables RT-PCR through modified ribonucleosides in RNA templates (e.g., naturally occurring post-transcriptional ribonucleosides in naturally occurring RNAs); (iii) the disclosed technology enables identification of post-transcriptional ribonucleosides through their mutational spectrum.

In some embodiments, the disclosed technology solves the problem of reverse transcribing the ribosomal RNA in its entirety from synthetic or environmental samples. The disclosed technology may be embodied in a kit that comprises all necessary components for performing RT-PCR of ribosomal RNA.

In some embodiments, the disclosed technology may be utilized to identify post-transcriptional ribonucleosides present in RNA templates from natural and synthetic samples. In some embodiments, the method comprises: (a) preparing a DNA molecule from the RNA template via reverse transcription by reacting a reaction mixture comprising: (i) a thermostable enzyme that comprises RNA-dependent DNA polymerase activity, (ii) the RNA template, (iii) one or more oligonucleotide primers that hybridize to the RNA template, and (iv) reagents for performing reverse transcription of the RNA template comprising deoxyribonucleotides which optionally are labeled; and (b) identifying the incorporated deoxyribonucleotides in the DNA molecule, (c) generating a mutation spectrum based on the incorporated deoxyribonucleotides at a position in the DNA molecule, (d) comparing the generated mutation spectrum to a reference mutation spectrum which is characteristic of the modified ribonucleoside, and (e) identifying the modified ribonucleoside in the RNA template at a position corresponding to the position in the DNA molecule. For example, where the reverse transcriptase of the disclosed methods incorporates a specific nucleotide in a nascent DNA molecule corresponding to a modified nucleoside present in an RNA template, the specific nucleotide may be utilized to identify the post-transcriptional ribonucleosides. As such, the disclosed technology can be embodied in a method or kit for identifying post-transcriptional ribonucleosides from the mutational spectrum they produce when reverse transcribed with the thermostable reverse transcriptases disclosed herein. In addition, without being bound by any theory or mechanism, the readthrough of post-transcriptionally modified ribonucleosides creates characteristic mutations in the nascent DNA strand. For example, in some embodiments, N¹-methylguanosine is readthrough by a polymerase, for example RTX polymerase, and produces a characteristic deoxycytosine in the nascent DNA strand where one of skill in the art would expect a deoxyguanosine to be present.

As used herein, “mutation spectrum” refers to the rate at which different types of mutations occur at different sites in the genome or at different sites in a particular polynucleotide, e.g. an RNA. Accordingly, one of skill in the art may use the mutation spectrum to identify the position of a putative post-transcriptionally modified ribonucleoside. Moreover, comparison of the generated mutation spectrum to the characteristic mutation spectra resulting from readthrough of post-transcriptional modifications will allow identification of the particular post-transcriptional modification in the polyribonucleotide.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and should not be interpreted to limit the scope of the claimed subject matter.

The ribosome is a two-subunit, macromolecular machine composed of RNA and proteins that carries out the polymerization of α-amino acids into polypeptides. Efforts to engineer ribosomal RNA (rRNA) deepen our understanding of molecular translation and provide opportunities to expand the chemistry of life by creating ribosomes with altered properties. Toward these efforts, reverse transcription PCR (RT-PCR) of the entire 16S and 23S rRNAs, which make up the 30S small subunit and 50S large subunit respectively, is important for isolating desired phenotypes. However, reverse transcription of rRNA is challenging due to extensive secondary structure and post-transcriptional modification. One key challenge is that existing commercial kits for RT-PCR rely on reverse transcriptases that lack the extreme thermostability and processivity found in many commercial DNA polymerases, which can result in subpar performance on challenging templates. Here, we develop methods employing a synthetic thermostable reverse transcriptase (RTX) to enable and optimize RT-PCR of the complete Escherichia coli 16S and 23S rRNAs. We also characterize the error rate of RTX when traversing the various post-transcriptional modifications of the 23S rRNA. We anticipate that this work will facilitate efforts to engineer the translation apparatus for synthetic biology.

### INTRODUCTION

Directed evolution of the ribosome and its associated translation factors has emerged as a promising opportunity to create new classes of enzymes, therapeutics, and materials with diverse genetically encoded chemistry (1-5). The key idea is that the ribosome can be repurposed to make proteins and polymers that selectively incorporate non-canonical monomers (6). To date, such efforts have incorporated a wide range of non-canonical α-(7), β-(8, 9), γ-(10-13), δ-, ε-, ξ-(13) D-(14, 15), aromatic (16-18), aliphatic (16, 19), malonyl(17), N-alkylated (20), cyclic (9, 13), and oligomeric amino acid analogues (10, 21, 22), among others. While incorporation of such diverse chemistries into peptides and proteins has facilitated exciting applications (e.g., macrocyclic foldamer-peptide drugs (23, 24)), there is poor compatibility with the natural translation apparatus for numerous classes of non-canonical monomers (e.g., backbone-extended amino acids) leading to incorporation inefficiencies. An especially challenging constraint is the ribosome, which has evolved to polymerize α-amino acids. Because its function is necessary for life, cell viability restricts the mutations that can be made to ribosomes.

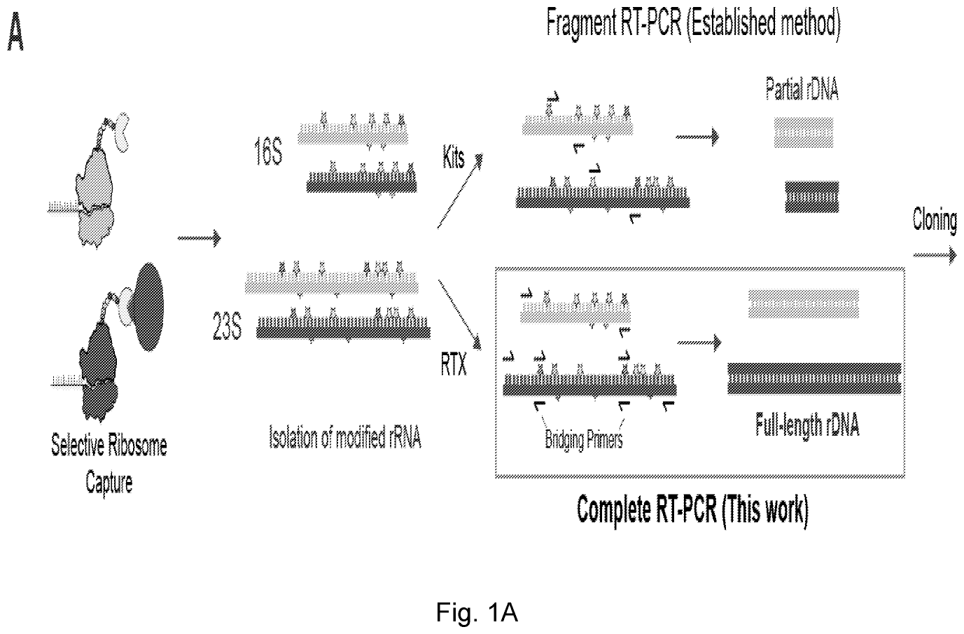

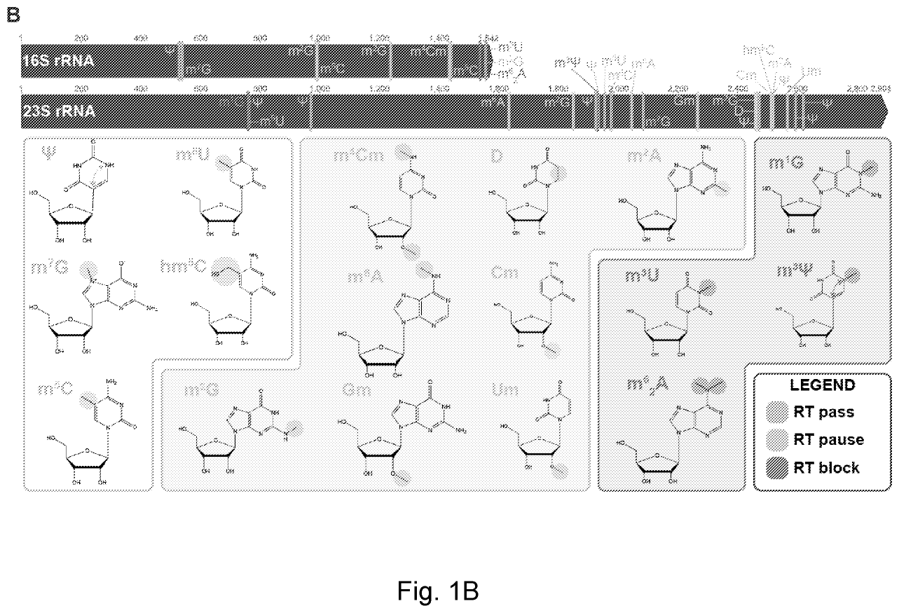

To overcome this compatibility challenge, new methods for engineering ribosomes have been developed in vivo and in vitro. In vivo, powerful positive/negative selections have been used to engineer orthogonal ribosomes (25, 26) and quadruplet decoding ribosomes (27), as well as ribosomes for β-amino acid incorporation (28) and D-amino acid incorporation (29, 30). In addition, the development of orthogonal tethered ribosomes has led to new functions inaccessible to the natural ribosome (31-35). In vitro, directed evolution of ribosomes using ribosome display offers the compelling advantages of flexibility and throughput, allowing access to lethal ribosomal genotype and larger rRNA variant libraries (36, 37). Despite these advances, key challenges remain. One such challenge to this approach is the recovery of full-length ribosomal cDNA (rDNA) from successful 16S and 23S rRNA sequences to enable directed evolution of the whole ribosome (FIG. 1A).

Typical methods for rRNA recovery, including 16S rRNA profiling broadly used for measuring microbial diversity in an environmental sample (38-45), rely on PCR of the rDNA (not rRNA) from genomic material in the sample. Additionally, these methods target only a small region of the 16S rRNA, as this is all that is required for microbial profiling. In contrast, RT-PCR methods for directed evolution of the ribosome for novel function would ideally allow recovery of the entire 16S and 23S rRNA, enabling diversification, selection, and recovery of disparate regions of the molecule (FIG. 1A). Unfortunately, rRNA is a challenging template for RT-PCR due to the presence of extensive secondary structure and post-transcriptional modifications (PTxMs), which can interfere with RT-PCR. These modifications can be categorized as being permissive to reverse-transcription, inducing pausing, or altogether blocking polymerization (46) (FIG. 1B). Blocking PTxMs generally disrupt the Watson-Crick interface of a base, while pausing PTxMs may weaken the base-pairing interaction or interfere with binding of the polymerase to ribose backbone. The 16S and 23S rRNAs of the E. coli ribosome contain 11 and 24 known modifications respectively (47) (Tables 1 and 2, below). For these reasons, recent efforts at in vitro directed evolution of the rRNA were limited to a small fragment of the 23S rRNA that did not contain any blocking PTxMs (36, 37).

In this study, we set out to develop a robust method for recovery of rDNA of the entire 16S rRNA and 23S rRNAs from translating ribosomes using a single enzyme. In this method, we leverage RT-PCR with a thermostable reverse transcriptase derived from the DNA polymerase KOD to identify regions of the 16S and 23S rRNAs which are intransigent to RT-PCR. Then, we optimized methods to bypass or polymerize through these challenging regions using RTX. Finally, we quantify the error rate when RTX polymerizes across various modified RNA nucleotides. We anticipate that our robust approach for RT-PCR of full-length 16S and 23S rRNA will facilitate efforts to engineer the ribosome.

Results

The goal of this study was to develop a methodology for producing consistent, robust RT-PCR product of full-length 16S and 23S rRNAs. We anticipated that this would be difficult because of the complex rRNA secondary structure resulting from the prevalence of post-transcriptional modifications (PTxMs) (46) and challenges surrounding the RT processivity. To start, we assessed the ability of a commercial reverse transcriptase kit; namely, SuperScript III, to achieve this goal using the manufacturer's recommended protocol. RNA from 70S ribosomes purified from E. coli MRE600 cells was used as the test template for RT-PCR experiments. Primers were designed with a high melting temperature to ensure proper annealing to the complex structure of the rRNA (Tables 3 and 4, below). As expected, this attempt failed to produce a robust single band product representing full-length rDNA.

We hypothesized that we could enable RT-PCR of full-length 16S and 23S rRNA by (i) employing a highly processive, thermostable reverse transcriptase and (ii) taking measures to enable the polymerase to either bypass or traverse the PTxMs present in rRNA. For the reverse transcriptase, we opted to utilize a recently developed synthetic RT (RTX) derived from the DNA polymerase KOD (48). Capable of recognizing both RNA-DNA and DNA-DNA duplexes and polymerizing DNA, this engineered polymerase performs both the RT step and subsequent PCR steps in a one-pot RT-PCR reaction. As a result, RTX had the potential to increase sensitivity by allowing multiple rounds of reverse-transcription (in contrast to commercial one-step kits, in which most of the RT enzyme is inactivated after one round), while also improving the quantity of the correct final product produced. To bypass PTxMs present in rRNA, we postulated that we could use bridging primers or long extension times.

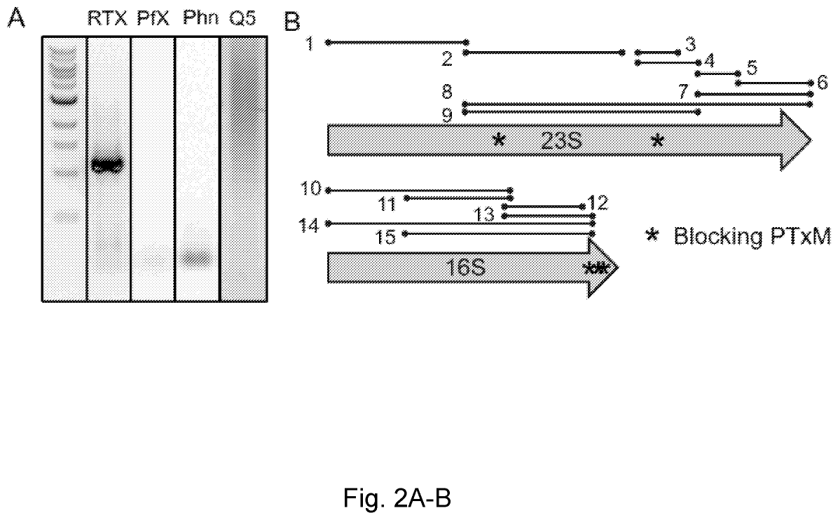

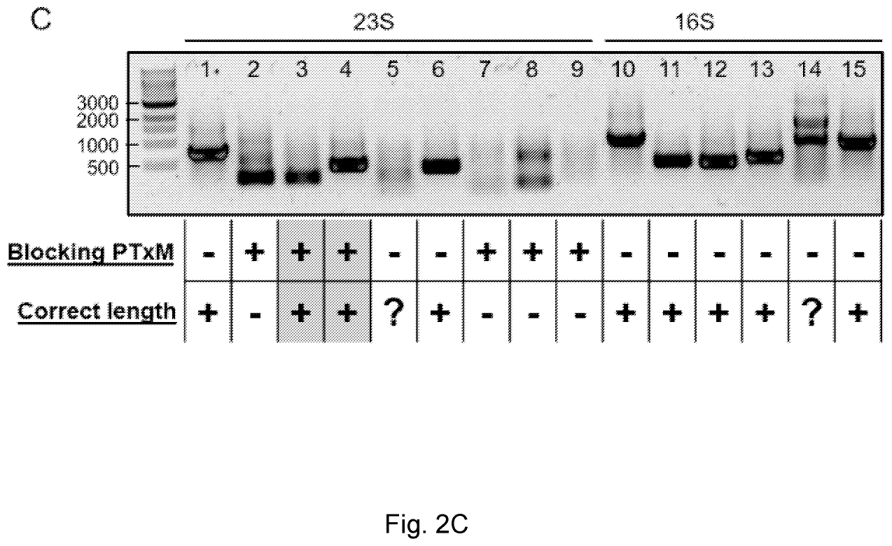

To assess the potential for RTX to RT-PCR of full-length rRNA, we first examined the ability of RTX to successfully polymerize various regions of the rRNA. Initially, RT-PCR was performed on an 1135 bp region spanning positions 784-1800 of the 23S rRNA which is known to lack blocking PTxMs. RTX produced a robust band at 1135 bp (FIG. 2A). As a control, we also carried out PCR reactions using three additional DNA polymerases (Pfx, Phusion, and Q5), which cannot reverse transcribe RNA. None of these polymerases produced bands of the correct size, indicating that our sample was free of contaminating genomic ribosomal cDNA.

We next assessed the ability of RTX to RT-PCR 9 regions of the 23S rRNA and 6 regions of the 16S rRNA known to have PTxMs (FIG. 2B). We started with the 23S rRNA. RT-PCR products spanning positions 1-770, 1867-2218, and 2535-2904 of the 23S rRNA, as well as nucleotides 1-980 and 945-1497 of the 16S rRNA were amenable to RT-PCR under standard conditions (68° C.-30 min, 35×(95° C.-30 sec, 68° C.-30 s/kb)). Longer 23S rRNA RT-PCR reactions spanning position 745 (1-methylguanosine), 1915 (3-methylpseudouridine), 2251 (2′-O-methylguanosine), and the region from 2498-2504 containing several PTxMs did not reliably produce full-length product in our initial tests (FIG. 2C, products 2, 5, 7-9). However, shorter targets spanning the blocking PTxM at position 1915 (FIG. 2C, products 3, 4) did produce products of correct size, suggesting that RTX may be capable of traversing blocking PTxMs.

Most of the 16S rRNA was amenable to RT-PCR, noting that the longest products used reverse primers that annealed just 5′ of three blocking PTxMs at the 3′ end of the gene (FIG. 1B). Single products were produced spanning positions 1-980 and 945-1497. The nearly full-length RT-PCR reaction spanning positions 1-1497 of the 16S rRNA produced some full-length product, but also a more dominant smaller off-product.

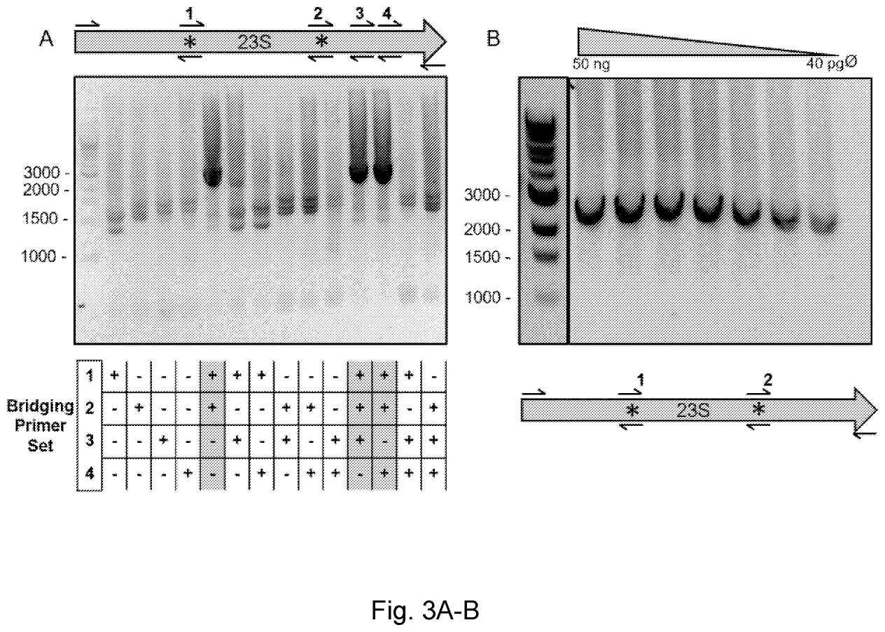

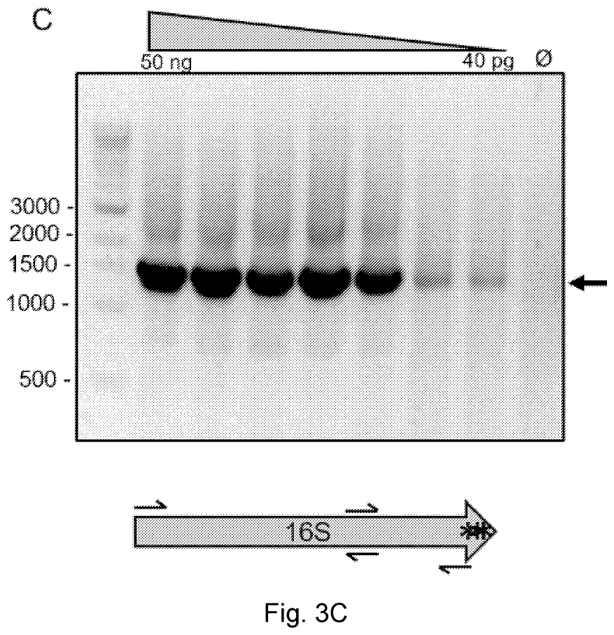

From these initial reactions, two strategies to recover full-length cDNA using 16S and 23S rRNAs as templates were attempted. The first RT-PCR strategy for achieving full-length ribosomal cDNA was to utilize “bridging primers” to anneal across problematic blocking or pausing post-transcriptional modifications. This would allow the production of constituent fragments of the 23S and 16S cDNA, which would be built into full-length products using overlap PCR in subsequent PCR cycles. Given previous results, positions 745 (1-methylguanosine), 1915 (3-methylpseudouridine), 2251 (2′-O-methylguanosine), and the region from 2498-2504 of the 23S rRNA were the prime candidates for bypassing with bridging primers (FIG. 3A, Table 2). Given the initial results for RT-PCR of the 16S rRNA (FIGS. 2B, 2C), we chose a single pair of bridging primers spanning modifications m²G and m⁵C at positions 966-967 (FIG. 3C, Table 1).

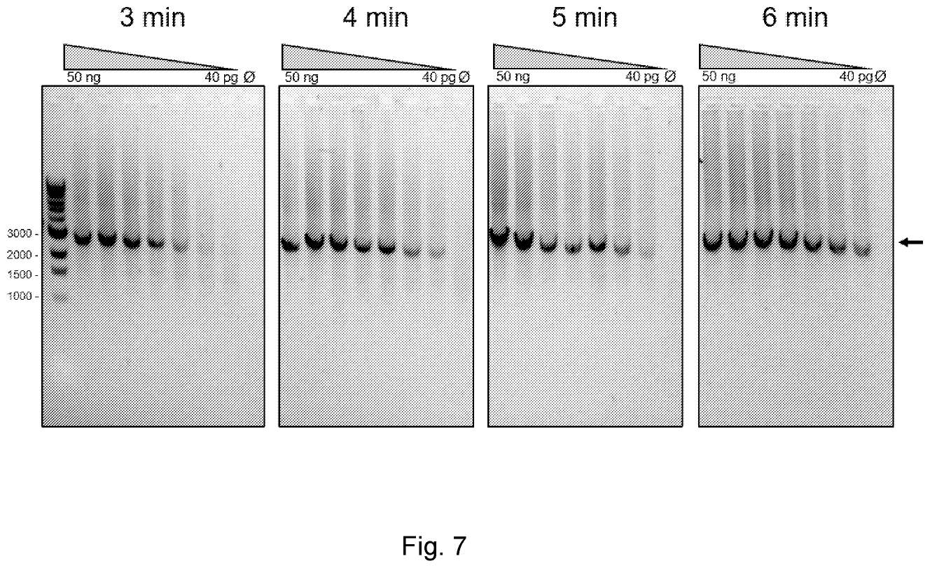

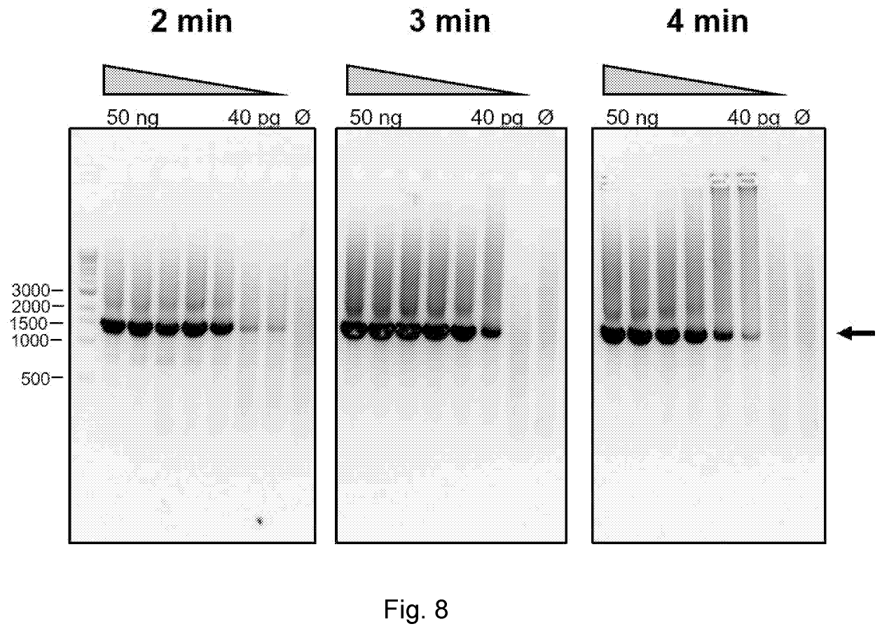

We next designed bridging primers to anneal across each of these PTxMs (Table 3). For the 23S rRNA, all possible combinations of these primers were tested to assess the minimal set of primers necessary to enable RT-PCR of the 23S rRNA. We found that including bridging primer sets 1 and 2 (corresponding to PTxMs at positions 745 and 1915 that disrupt Watson-Crick base pairing) were the minimal set which enabled robust RT-PCR of the 23S rRNA (FIG. 3A). Using this primer set for the 23S rRNA and the single set designed for the 16S rRNA, we found that this strategy enabled recovery of full-length rDNA using elongation steps of between 3 and 6 minutes for the 23S rRNA (FIG. 7), and between 2 and 4 minutes with the 16S rRNA (FIG. 8). Optimal results were achieved from the protocol with 6-minute elongation for the 23S rRNA (FIG. 3B) and the 3-minute elongation for the 16S rRNA (FIG. 3C), enabling recovery of full-length rDNA from down to 40 pg of rRNA in each case.

While the bridging primer strategy allowed production of the correct product, RT-PCR using bridging primers necessarily entails loss of genotype fidelity, due to recombining of the regions broken up by the bridging primers. This may be undesirable for ribosome engineering applications. Additionally, we were only able to recover cDNA from 97% of the 16S rRNA gene using bridging primers, since the three blocking PTxMs of the 16S rRNA are so proximal to the 3′ end of the RNA. We wondered if there might be alternative strategies for achieving full-length RT-PCR of the 23S and 16S rRNAs that did not require bridging primers.

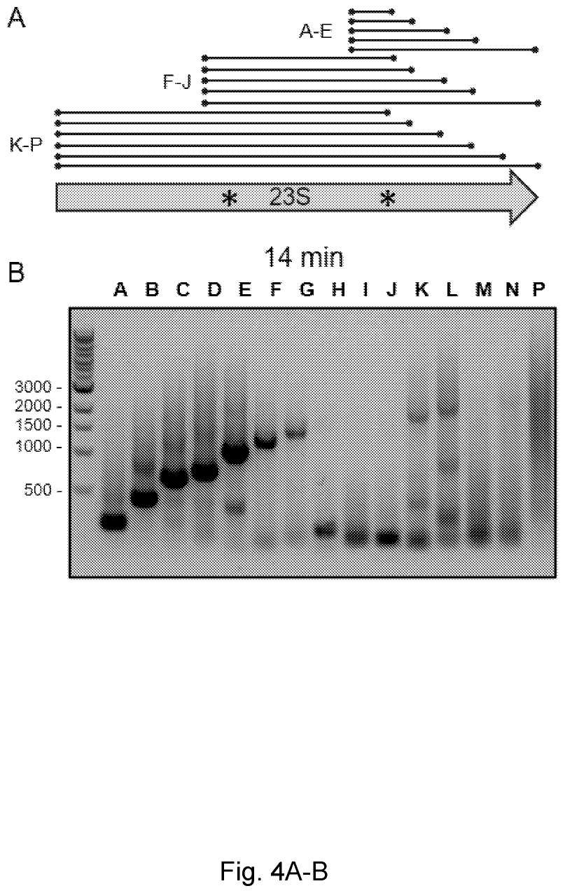

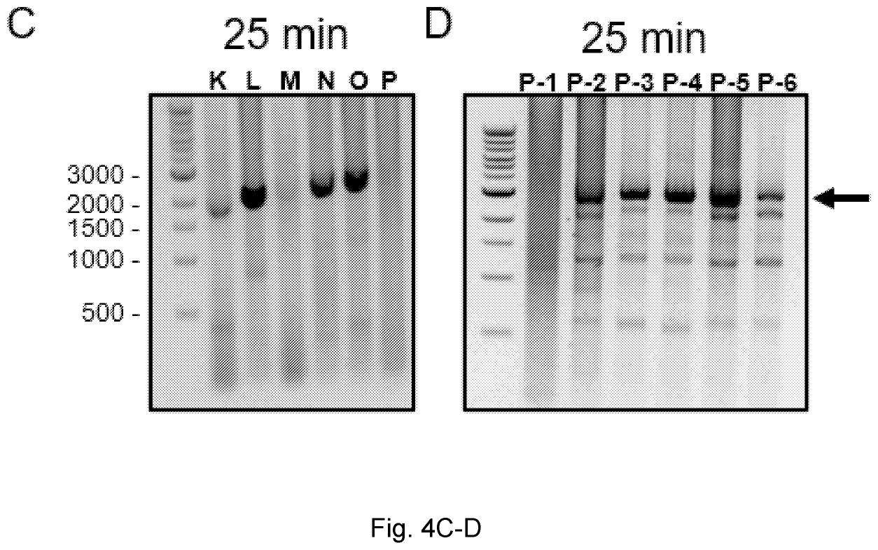

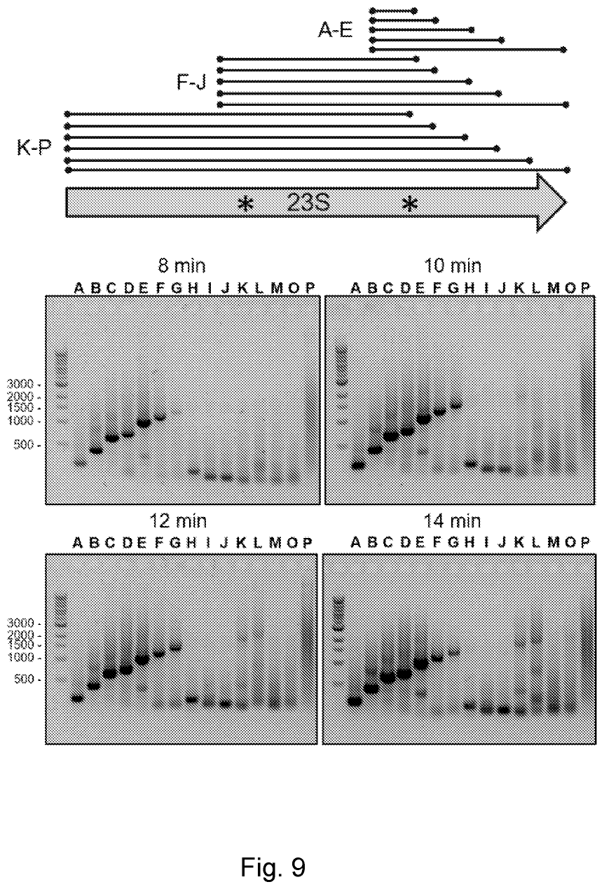

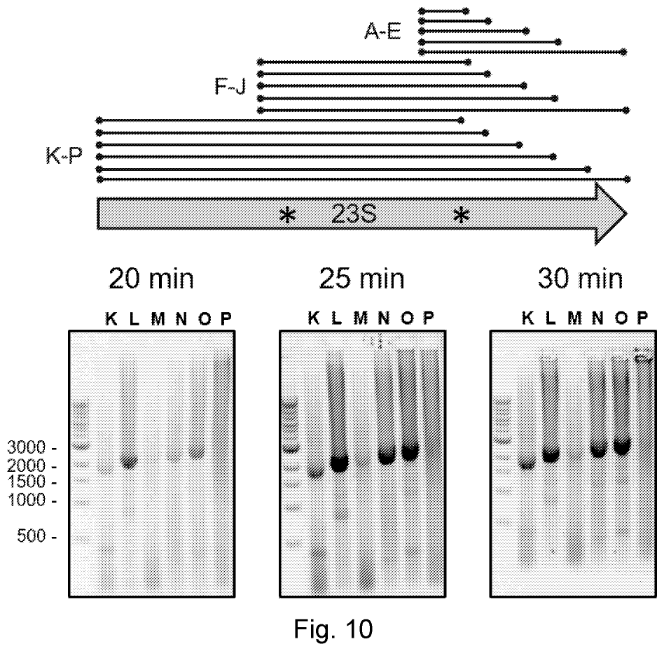

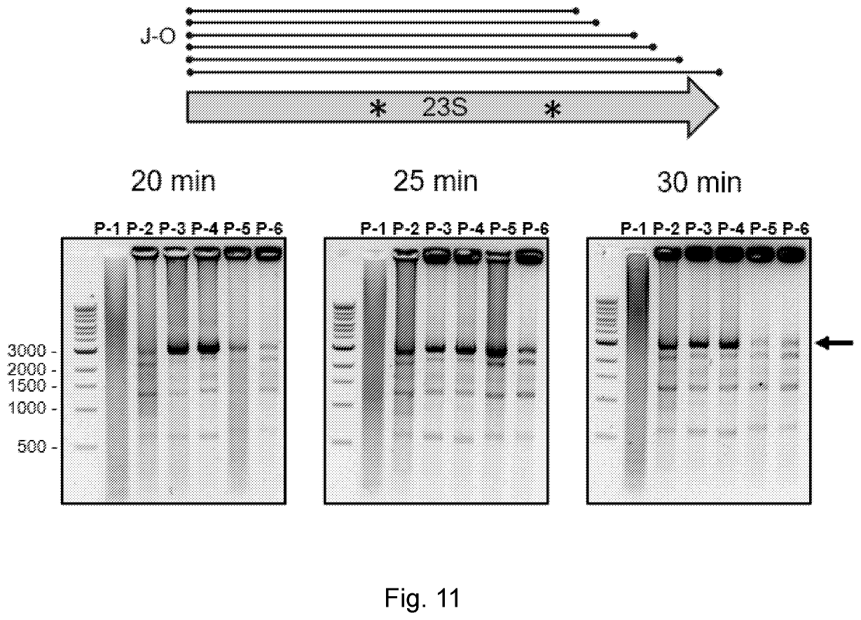

As an alternative, we assessed the possibility of RTX read-through of PTxMs by testing for long extension times. To do so, we attempted RT-PCR of the 23S rRNA with products spanning various regions of increasing length with elongation steps of 8, 10, 12, and 14 minutes (FIG. 4A, FIG. 9, Table 4). We observed improved production of desired products with increasing extension duration. In particular, products K and L, comprising 2000 nucleotides and spanning the two blocking PTxMs we identified earlier (FIG. 2), benefited from increasing extension time, with little or no observed product in the 8-minute extension step and a clear observable product in the 14-minute condition (FIG. 4B, FIG. 9). These results encouraged us to try even more extreme extension durations to achieve full-length products of these templates. Given our previous success using a forward primer at the 5′ extremity of the 23S rRNA, we held this primer constant and attempted RT-PCRs on products ranging from 2055-2904 bases in length with extension steps of 20, 25, and 30 minutes (FIG. 10). We achieved robust products up to 2675 bases in length, with 25- and 30-minute extension steps yielding the strongest bands (FIG. 4C). Our failure once again to achieve full-length products for the 23S rRNA despite the absence of blocking or pausing post-transcriptional modifications between positions 2675 and 2904 led us to implicate a problematic reverse primer in this RT-PCR, and thus to attempt a variety of primers at the 3′ extremity of the 23S rRNA. Using a variety of reverse primers and varying elongation times, we were able to achieve full-length product of the 23S rRNA (FIG. 4D, FIG. 11, Table 4) Notably, however, these RT-PCR reactions contained both low and high molecular weight aberrant products.

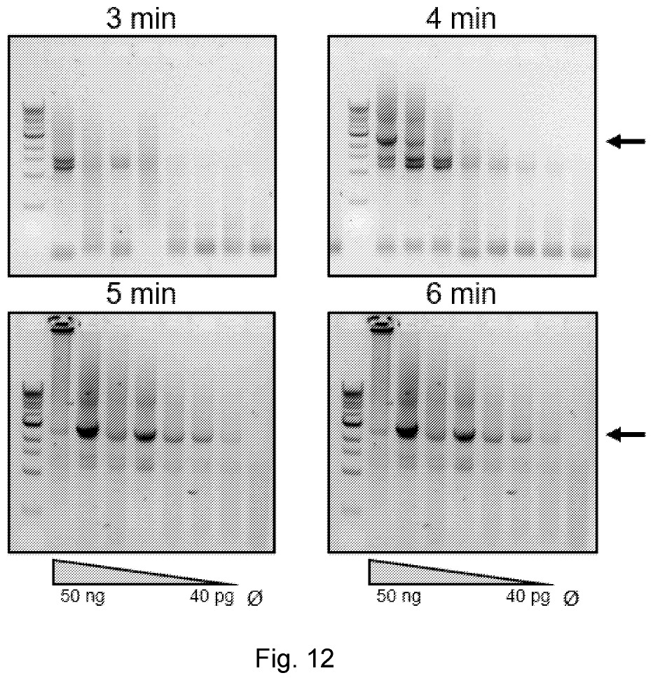

While long extension times enabled production of full-length 23S rDNA product, we hypothesized that extension times of this length would not be necessary for all the cycles of a PCR reaction, since the initial 30-minute cycles would produce template cDNA for future cycles which should be more amenable to PCR. To test whether shortening these steps reduced off-product while retaining full-length product, we performed reactions with one 30-minute extension step, followed by remaining cycles with, 3-, 4-, 5-, or 6-minute extension steps for the 23S rRNA (FIG. 12). We found that 5-minute or longer extension steps were still required for the 23S rRNA. This suggests that RTX requires more time to traverse the difficult 23S rRNA template than typical templates, even when it has already been reverse-transcribed into cDNA by the first long RT cycles.

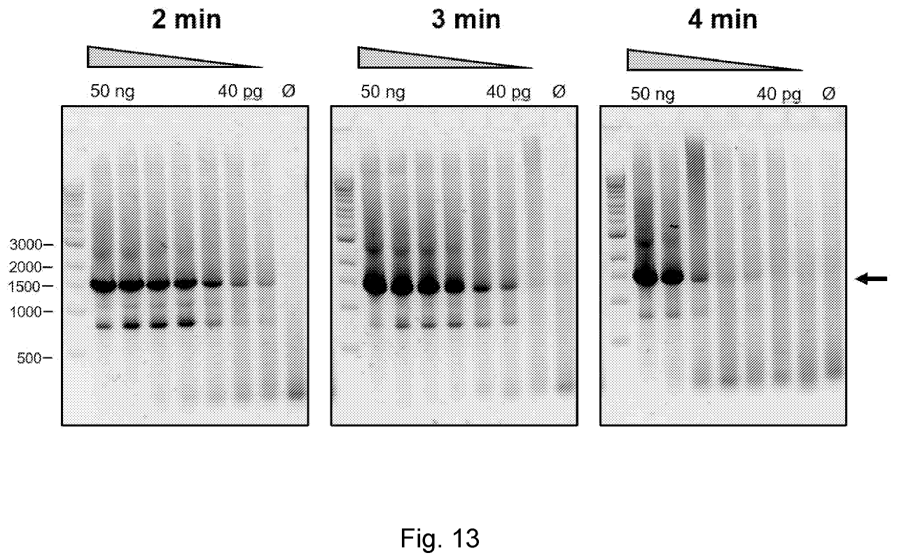

Using the information gained from optimization of the 23S rRNA RT-PCR, we attempted RT-PCR of the 16S rRNA using no bridging primers, an initial 30-minute extension step, and 2-, 3-, or 4-minute elongation steps while cycling. While we were able to produce the correct product under all cycling conditions, we found that RT-PCR of the 16S rRNA without bridging primers generally resulted in production of many off-products as well, likely requiring extraction of the correct band for downstream applications (FIG. 13).

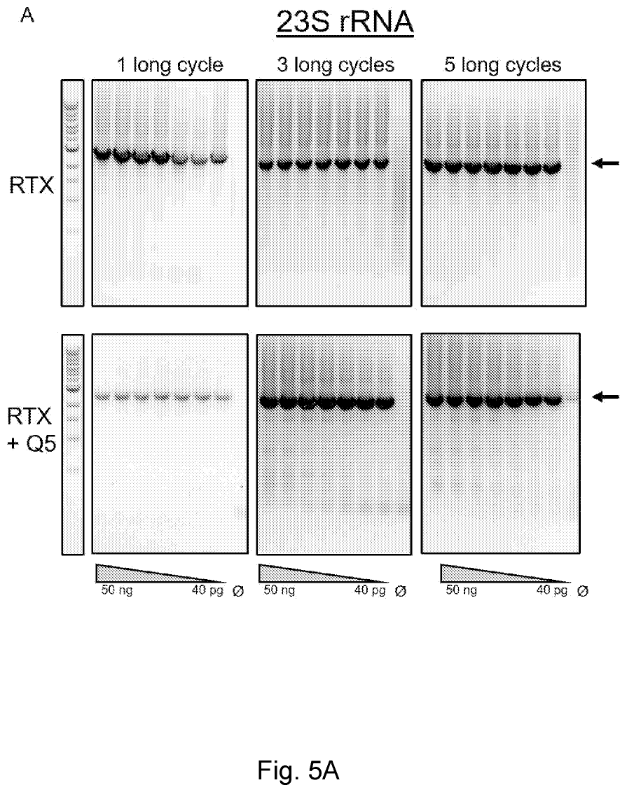

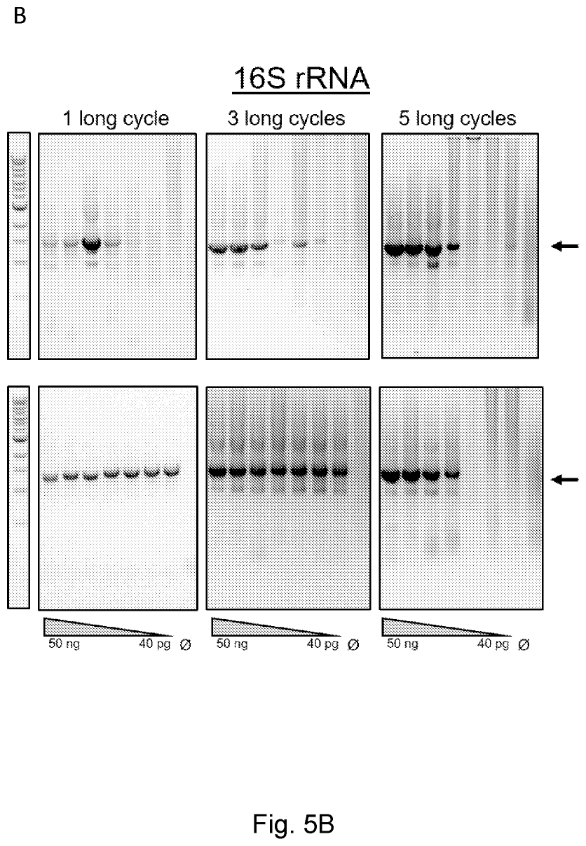

Finally, we attempted a strategy mimicking one-step RT-PCR kits, which contain a reverse transcriptase and DNA polymerase in an enzyme mix to enable RT-PCR. We hypothesized that the added benefit of thermostability of RTX would enable multiple initial RT steps before the proofreading DNA polymerase takes over (Materials and Methods). Results were comparable for the RTX alone and RTX+Q5 reactions, indicating that this dual enzyme strategy did not improve amplification of the 23S rRNA when using RTX (FIG. 5A). However, this strategy did result in more reliable production of full-length 16S cDNA (FIG. 5B).

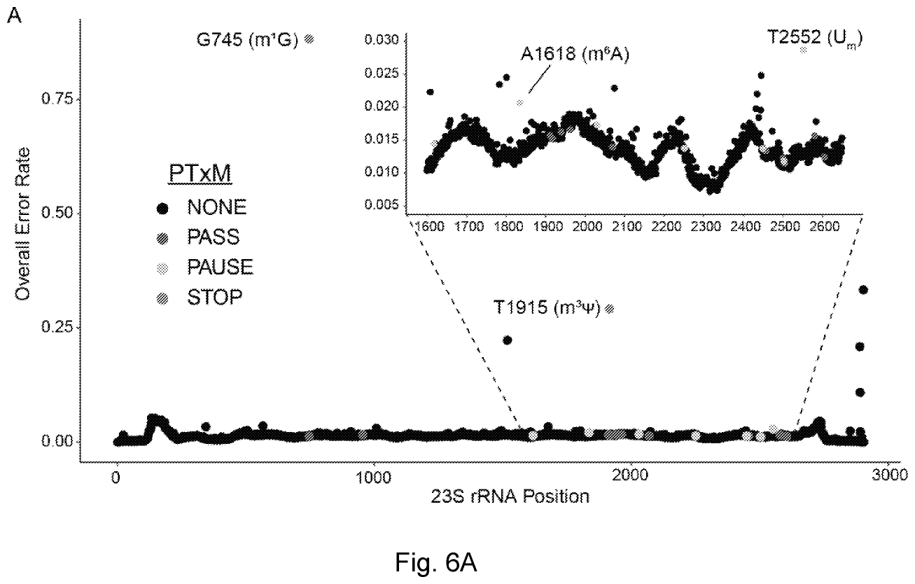

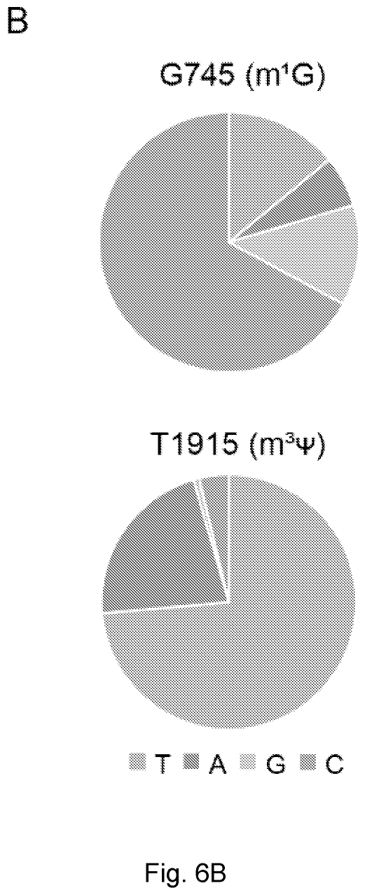

The base modifications present in rRNA are known to cause elevated error rates in reverse-transcription reactions, a fact which has been leveraged recently to identify modified nucleotides from transcriptomic data (49-52). Since we used the version of RTX lacking proofreading exonuclease function, we anticipated that using this enzyme to polymerize through modified nucleotides—especially those canonically thought to block polymerization—would result in elevated error rates at these positions. To assess the error rate of RTX across the 23S rRNA, cDNA from a full-length RT-PCR reaction of the 23S rRNA lacking bridging primers was deep sequenced, and divergence of the observed base from wild-type at each position was quantified (FIG. 6A). As expected, the two blocking PTxMs in the 23S rRNA (m¹G and m³Ψ at positions 745 and 1915, respectively) had elevated misincorporation rates of 88.2% (s.d.=0.003, n=4) and 29.2% (s.d.=0.002, n=4) respectively. These two modifications cause widely differing mutational spectra as well which were consistent across 4 replicates, with “C” and “A” as the dominant missense mutation for positions 745 and 1915, respectively (FIG. 6B). While most other permissive and pausing modifications resulted in error rates comparable to the surrounding region of the 23s rRNA, modifications m⁶A and Uₘ at positions 1618 and 2552 also had elevated rates of missense mutation (FIG. 6A, inset). This is consistent with previous work showing that reverse transcription across m⁶A results in an elevated error rate (50). Interestingly, a number of other positions which are not reported to be modified display an elevated mutation rate above the trend for that region of the 23s rRNA. It is unclear whether these positions are the result of sequencing artifacts or reflect real molecular heterogeneity or modification, and further investigation may be necessary to discern the source of the elevated rate of mismatch mutations at these positions (49, 51).

### DISCUSSION

We developed and optimized methodologies to achieve full-length RT-PCR of the 23S and 16S rRNAs. Specifically, we leveraged two strategies: (i) bridging primers that anneal over PTxMs to bypass them and (ii) long extension steps to polymerize through these positions. Taken together, these methods should facilitate efforts to study and engineer mutant ribosomes.

We present two strategies since they each have advantages and disadvantages. The use of bridging primers has the advantage of a robust correct product and fewer off-products for both the 23S and 16S rRNAs. Additionally, for directed evolution studies, recombination of distal regions of the rRNA that arise in this approach would add new genetic diversity of the ribosome pool under selection. The disadvantage of this approach is that the bridging primers result in a loss of genotype fidelity. RT-PCR using a long extension step to polymerize through PTxMs may be the more desirable strategy in situations where the genotype of distal regions of the ribosome must be preserved. However, the RT-PCR itself is less robust to synthesis of a single target product. Moreover, the RTX enzyme used here has an elevated error rate when polymerizing through blocking PTxMs. Studying the effect of removing PTxMs individually and in combination would help elucidate the role of these modifications in translation function.

Up to now, efforts to engineer ribosomes in vitro that rely on RT-PCR have been limited by the inability to recover the entire 16S and 23S rRNA (36, 37). Looking forward, the methods reported here should enable diversification, selection, and recovery of disparate regions of the ribosome, setting the stage for new directions at the interface of chemical and synthetic biology.

Materials and Methods:

Purification of RTX

pET_RTX_(exo-) (Addgene #102786) was transformed into BL21 (DE3) cells and inoculated in LB overnight. Cells were diluted at a ratio of 1:250 and induced at mid-log phase with 1 mM IPTG. Protein was expressed at 18° C. overnight. Cells were pelleted at 5000× g for 10 minutes and resuspended in Buffer A (10 mM phosphate, 100 mM NaCl, 0.1 mM EDTA, 1 mM DTT, 10% glycerol, pH 7). Cells were lysed in an Avestin Emulsiflex-B15 homogenizer and benzonase (Millipore) was added and incubated for 1 hour at 37° C. to degrade cellular nucleic acids. Cell lysates were then heated at 85° C. for 25 minutes, cooled on ice, and cell debris was pelleted. Cleared cell lysate was incubated with Qiagen nickel-NTA resin for 1 hour at 4° C. Resin was washed 5 times with wash buffer (50 mM NaH2PO₄, 300 mM NaCl, 6 mM BME, pH 8) with 10 mM imidazole, and 3 times with 20 mM imidazole. Protein was eluted using the same buffer composition plus 200 mM imidazole. Purified protein was dialyzed into storage buffer (50 mM Tris-HCl, 50 mM KCl, 0.1 mM EDTA, 1 mM DTT, 0.1% Tween20, 50% glycerol, pH 8.0), concentrated to 0.4 mg/mL using Centriprep centrifugal filters, 3,000 MWCO, and stored in aliquots at −80° C.

Purification of Ribosomal RNA

Lysate generated from MRE600 cells harvested at OD600 of 0.5 was centrifuged for 30 minutes at 30,000×g to remove cellular debris. Supernatant was layered in a 1:1 volumetric ratio on a high-salt sucrose cushion containing 20 mM Tris-HCl pH 7.2, 500 mM NH₄Cl, 10 mM MgCl₂, 0.5 mM EDTA, 6 mM BME, and 37.7% sucrose in a Ti70 ultracentrifuge tube and centrifuged at 4° C. and 90,000×g overnight. The next morning the supernatant was removed and spun at 4° C. and 150,000×g for an additional 3 hours. The supernatant was removed, and the remaining pellet, consisting primarily of 70S ribosomes, was gently washed with Buffer C (10 mM Tris-OAc (pH=7.5 at 4° C.), 60 mM NH₄Cl, 7.5 mM Mg(OAc)₂, 0.5 mM EDTA, 2 mM DTT) until the pellet was glassy. This pellet was then resuspended in Buffer C for at least 2 hours shaking at 4° C. To precipitate RNA, two volumes of glacial acetic acid were added to the sample and incubated for 10 minutes at 4° C. The sample was then centrifuged at 16,000 g for 30 minutes to pellet rRNA. Pelleted rRNA resuspended in nuclease-free water and purified using a Qiagen RNEasy Mini Kit, eluted using nuclease-free water, and stored in aliquots at −80° C.

Reverse Transcription PCR Using RTX

RT-PCR using RTX was carried out in 1× RTX buffer (60 mM Tris-HCl (pH 8.4), 25 mM (NH₄)₂SO₄, 10 mM KCl), 200 μM dNTPs, 1 mM MgSO₄, 1 M betaine, 0.4 μg RTX exo-). Initially, the cycling conditions indicated in the original manuscript were utilized: 68. C-30 min, 35× (95. C-30 sec, 68. C-30 s/kb). Following optimization, the cycling parameters were adjusted to 2 min/kb for the 23S rRNA. Following RT-PCR, 5 μL of each reaction was run on a 1% agarose gel in TAE running buffer to assess success of the RT-PCR reaction.

Two Enzyme RT-PCR Using RTX and Q5 Polymerase

One-step mixes featuring either RTX buffer or Q5 buffer were attempted, with poor results from both buffer conditions, likely owing to the buffers' substantially different pH and salt composition. To allow each enzyme to perform under more ideal conditions, we assembled 25 uL RT reactions to amplify the 23S rRNA in RTX buffer, allowing 1, 3, or 5 long (30 min) extension steps. Then, 5 uL of this reaction was removed, and mixed with 20 uL of Q5 reaction mix. Each reaction (RTX and Q5) was then cycled under the optimal conditions for its respective enzyme.

A gradient of rRNA concentrations was cycled for 1, 3, or 5 cycles in an 25 RTX RT-PCR reaction using the reaction formulation described above and the cycling conditions (95° C.-30 sec, 68° C.-25 min). After completion of the RT-PCR cycles, 5 μL of this reaction was added to a 25 μL Q5 polymerase reaction (NEB #M0491) and cycled using standard reaction conditions. The RTX reactions were also returned to the thermocycler and cycled for an addition 35 cycles using the protocol 35× (95° C.-30 sec, 68° C.-25 min). 5 μL of each of these reactions were run on a 1% TAE agarose gel to assess product formation.

Deep Sequencing Analysis of Reverse Transcribed cDNA

RT-qPCR of the 23S and 16S rRNAs was performed using only external primers and the protocol: 68° C.-30 min, 35× (95° C.-30 sec, 68° C.-X min), where X=6 minutes and 2 minutes, respectively. The 23S cDNA was processed for sequencing using the NEXTNext Ultra II FS DNA DNA Library Prep Kit and barcoded using NEBNEXT Multiplex Oligos for Illumina Primers Sets 1 and 2. Samples were submitted for sequencing at Genewiz using the Illumina HiSeq 4000 platform with 2×150 bp paired end reads. Raw sequencing reads were filtered using sickle (Available at https://github.cominajoshi/sickle) and merged using PANDAseq (53). Paired reads were then aligned to the reference sequence using BWA (54). SAM files were converted to BAM files and sorted, and pileup files were generated all using SAMtools (55). A table of nucleotide by nucleotide variation was generated using the pileup2acgt function in Sequenza (56). Statistical analysis of these data tables and figure generation was performed using R. For error rate analysis, positions of the 23S rRNA which are polymorphic in MRE600 E. coli were excluded from analysis, as heterogeneity at these positions reflects polymorphism and not RTX error rate.

Supplementary Information

TABLE 1
Post-transcriptional modifications of the E. coli 16S rRNA.
This table lists the positions of each modification, the type of
modification made, and the abbreviation commonly used to denote
this modification, which appear in FIG. 1. This information is
taken from The RNA Modification Database
(mods.rna.albany.edu/Introduction/Ribosomal-RNA).

| rRNA | Position | Modification | Abbreviation |
|---|---|---|---|
| 16S | 516 | Pseudouridine | Ψ |
| 16S | 527 | N⁷-methylguanosine | m⁷G |
| 16S | 966 | N²-methylguanosine | m²G |
| 16S | 967 | 5-methylcytosine | m⁵C |
| 16S | 1207 | N²-methylguanosine | m²G |
| 16S | 1402 | N⁴,2′-O-methylcytosine | m⁴C |
| 16S | 1407 | 5-methylcytosine | m⁵C |
| 16S | 1498 | N³-methyluridine | m³U |
| 16S | 1516 | N²-methylguanosine | m²G |
| 16S | 1518 | N⁶,N⁶-dimethyladenosine | m⁶₂A |
| 16S | 1519 | N⁶,N⁶-dimethyladenosine | m⁶₂A |

TABLE 2
Post-transcriptional modifications of the E. coli 23S rRNA.
This table lists the positions of each modification, the type of
modification made, and the abbreviation commonly used to denote
this modification, which appear in FIG. 1. This information
is taken from The RNA Modification Database
(mods.rna.albany.edu/Introduction/Ribosomal-RNA).

| rRNA | Position | Modification | Abbreviation |
|---|---|---|---|
| 23S | 745 | N¹-methyllguanosine | m¹G |
| 23S | 746 | Pseudouridine | Ψ |
| 23S | 747 | 5-methyluridine | m⁵U |
| 23S | 955 | Pseudouridine | Ψ |
| 23S | 1618 | N⁶-methyladenosine | m⁶A |
| 23S | 1835 | N²-methylguanosine | m²G |
| 23S | 1911 | Pseudouridine | Ψ |
| 23S | 1915 | N³-methylpseudouridine | m³Y |
| 23S | 1917 | Pseudouridine | Ψ |
| 23S | 1939 | 5-methyluridine | m⁵U |
| 23S | 1962 | 5-methylcytosine | m⁵C |
| 23S | 2030 | N⁶-methyladenosine | m⁶A |
| 23S | 2069 | N⁷-methylguanosine | m⁷G |
| 23S | 2251 | 2′-O-methylguanosine | Gm |
| 23S | 2445 | N²-methylguanosine | m²G |
| 23S | 2449 | Dihydrouridine | D |
| 23S | 2457 | Pseudouridine | Ψ |
| 23S | 2498 | 2′-O-methylcytosine | Cm |
| 23S | 2501 | 5-hydroxymethylcytidine | hm⁵C |
| 23S | 2503 | N²-methyladenosine | m²A |
| 23S | 2504 | Pseudouridine | Ψ |
| 23S | 2552 | 2′-O-methyluridine | Um |
| 23S | 2580 | Pseudouridine | Ψ |
| 23S | 2605 | Pseudouridine | Ψ |

TABLE 3
Primers used in initial RT-PCR tests of 23S rRNA.

| Products | | | |
|---|---|---|---|
| (FIG. 2) | Primer Name | Sequence | Notes |
| 1 | 23S_rRNA_for | GGTTAAGCGACTAAGCGTACACG | |
| | | GTGGATGCC (SEQ ID NO: | |
| | | 1) | |
| 2, 8, 9 | 23S_rRNA_784_for | GGAGGACCGAACCGACTAATGTT | Forward |
| | | GAAAAATTAGCGGATGACTTGTG | bridging |
| | | GCTGGGGGTGAAAG (SEQ ID | primer for |
| | | NO: 2) | position 745 |
| 1 | 23S_rRNA_708_rev | AAGTCATCCGCTAATTTTTCAAC | Reverse |
| | | ATTAGTCGGTTCGGTCCTCCAGT | bridging |
| | | TAGTGTTACCCAAC (SEQ ID | primer for |
| | | NO: 3) | position 745 |
| 3, 4 | 23S_rRNA_1867_for | ACGGTGTGACGCCTGCCCGGTGC | |
| | | CGGAAGGTTAATTGATGGGGTTA | |
| | | G (SEQ ID NO: 4) | |
| 2 | 23S_rRNA_1800_rev | ATCAATTAACCTTCCGGCACCGG | |
| | | GCAGGCGTCACACCGTATACGTC | |
| | | CACTTTCGTGTTTG (SEQ ID | |
| | | NO: 5) | |
| | 23s_rRNA_1954_for | CGGTAAACGGCGGCCGTAACTAT | Forward |
| | | AACGGTCCTAAGGTAGCGAAATT | bridging |
| | | CCTTGTCGGGTAAG (SEQ ID | primer for |
| | | NO: 6) | position 1915 |
| 3 | 23S_rRNA_1995_rev | TCTTGCCGCGGGTACACTGCATC | |
| | | TTCACAGCGAGTTCAATTTCACT | |
| | | GAGTCTCGGGTGGA (SEQ ID | |
| | | NO: 43) | |
| | 23S_rRNA_1881_rev | AGGACCGTTATAGTTACGGCCGC | Reverse |
| | | CGTTTACCGGGGCTTCG (SEQ | bridging |
| | | ID NO: 7) | primer for |
| | | | position 1915 |
| 5, 7 | 23S_rRNA_2282_for | CTGGGGCGGTCTCCTCCTAAAGA | Forward |
| | | GTAACGGAGGAG (SEQ ID | bridging |
| | | NO: 8) | primer for |
| | | | position 2251 |
| 4, 9 | 23S_rRNA_2218_rev | CCGTTACTCTTTAGGAGGAGACC | Reverse |
| | | GCCCCAGTCAAACTACCCACCAG | bridging |
| | | ACACTGTCCGCAAC (SEQ ID | primer for |
| | | NO: 9) | position 2251 |
| 6 | 23s_rRNA_2535_for | GCACCTCGATGTCGGCTCATCAC | Forward |
| | | ATCCTGGGGCTGAAGTAG (SEQ | bridging |
| | | ID NO: 10) | primer for |
| | | | region |
| | | | 2498-2504 |
| 5 | 23s_rRNA_2465_rev | CCAGGATGTGATGAGCCGACATC | Reverse |
| | | GAGGTGCCAAACACCGCCGTCGA | bridging |
| | | TATGAACTCTTGGG (SEQ ID | primer for |
| | | NO: 11) | region |
| | | | 2498-2504 |
| 6, 7, 9 | 23s_rRNA_end_rev | AAGGTTAAGCCTCACGGTTCATT | |
| | | AGTACCGGTTAGCTC (SEQ ID | |
| | | NO: 12) | |
| 10, 14 | 16s_rRNA_42_for | AAATTGAAGAGTTTGATCATGGC | |
| | | TCAGATTGAACGCTGGCGG | |
| | | (SEQ ID NO: 13) | |
| 11, 15 | 16S_rRNA_543_for | TGCCAGCAGCCGCGGTAATACGG | |
| | | AGGGT (SEQ ID NO: 14) | |
| 12, 13 | 16S_rRNA_1002_for | GGAGCATGTGGTTTAATTCGATG | Bridging |
| | | CAACGCGAAGAACCTTACCTGGT | primer for |
| | | CTTGACATCCACG (SEQ ID | 16S rRNA |
| | | NO: 15) | |
| 10, 11 | 16S_rRNA_931_rev | GGTTCTTCGCGTTGCATCGAATT | Bridging |
| | | AAACCACATGCTCCACCGCTTGT | primer for |
| | | GCGG (SEQ ID NO: 16) | 16S rRNA |
| 12, 13, | 16S_rRNA_1461_rev | CGACTTCACCCCAGTCATGAATC | |
| 14, 15 | | ACAAAGTGGTAAGC (SEQ ID | |
| | | NO: 17) | |
| | 16S_rev | TAAGGAGGTGATCCAACCGCAGG | |
| | | (SEQ ID NO: 18) | |

TABLE 4
Primers used to optimized RT-PCR of 23S rRNA without
bridging primers.

| | Forward | | Anticipated |
|---|---|---|---|
| Product | Primer | Reverse Primer | Size |
| A | ACGGTGTG | TCTTGCCGCGGGTACACTGCATCTTCACA | 234 |
| | ACGCCTGC | GCGAGTTCAATTTCACTGAGTCTCGGGTG | |
| | CCGGTGCC | GA (SEQ ID NO: 20) | |
| | GGAAGGTT | | |
| | AATTGATG | | |
| | GGGTTAG | | |
| | (SEQ ID | | |
| | NO: 19) | | |
| B | | CCGTTACTCTTTAGGAGGAGACCGCCCCA | 457 |
| | | GTCAAACTACCCACCAGACACTGTCCGCA | |
| | | AC (SEQ ID NO: 21) | |
| C | | CCAGGATGTGATGAGCCGACATCGAGGTG | 704 |
| | | CCAAACACCGCCGTCGATATGAACTCTTG | |
| | | GG (SEQ ID NO: 22) | |
| D | | CGACGTTCTAAACCCAGCTC (SEQ ID | 775 |
| | | NO: 23) | |
| E | | GGCGTTGTAAGGTTAAGCCTCAC (SEQ | 1084 |
| | | ID NO: 24) | |
| F | GGAGGACC | TCTTGCCGCGGGTACACTGCATCTTCACA | 1330 |
| | GAACCGAC | GCGAGTTCAATTTCACTGAGTCTCGGGTG | |
| | TAATGTTG | GA (SEQ ID NO: 26) | |
| | AAAAATTA | | |
| | GCGGATGA | | |
| | CTTGTGGC | | |
| | TGGGGGTG | | |
| | AAAG | | |
| | (SEQ ID | | |
| | NO: 25) | | |
| G | | CCGTTACTCTTTAGGAGGAGACCGCCCCA | 1553 |
| | | GTCAAACTACCCACCAGACACTGTCCGCA | |
| | | AC (SEQ ID NO: 27) | |
| H | | CCAGGATGTGATGAGCCGACATCGAGGTG | 1800 |
| | | CCAAACACCGCCGTCGATATGAACTCTTG | |
| | | GG (SEQ ID NO: 28) | |
| I | | CGACGTTCTAAACCCAGCTC (SEQ ID | 1871 |
| | | NO: 29) | |
| J | | GGCGTTGTAAGGTTAAGCCTCAC (SEQ | 2180 |
| | | ID NO: 30) | |
| K | GGTTAAGC | TCTTGCCGCGGGTACACTGCATCTTCACA | 2054 |
| | GACTAAGC | GCGAGTTCAATTTCACTGAGTCTCGGGTG | |
| | GTACACGG | GA (SEQ ID NO: 31) | |
| | TGGATGCC | | |
| | (SEQ ID | | |
| | NO: 42) | | |
| L | | CCGTTACTCTTTAGGAGGAGACCGCCCCA | 2277 |
| | | GTCAAACTACCCACCAGACACTGTCCGCA | |
| | | AC (SEQ ID NO: 32) | |
| M | | CCAGGATGTGATGAGCCGACATCGAGGTG | 2524 |
| | | CCAAACACCGCCGTCGATATGAACTCTTG | |
| | | GG (SEQ ID NO: 33) | |
| N | | CGACGTTCTAAACCCAGCTC (SEQ ID | 2595 |
| | | NO: 34) | |
| O | | CCACTCCGGTCCTCTCGTACTAG (SEQ | 2674 |
| | | ID NO: 35) | |
| P-1 | | GGCGTTGTAAGGTTAAGCCTCAC (SEQ | 2904 |
| | | ID NO: 36) | |
| P-2 | | AAGGTTAAGCCTCACGGTTCATTAG | 2904 |
| | | (SEQ ID NO: 37) | |
| P-3 | | AAGGTTAAGCCTCACGGTTCATTAGTACC | 2904 |
| | | GG (SEQ ID NO: 38) | |
| P-4 | | AAGGTTAAGCCTCACGGTTCATTAGTACC | 2904 |
| | | GGTTAGCTC (SEQ ID NO: 39) | |
| P-5 | | GGTTCATTAGTACCGGTTAGCTCAACGCA | 2889 |
| | | TCGCTGCG (SEQ ID NO: 40) | |
| P-6 | | GCTCAACGCATCGCTGCGCTTACACACCC | 2870 |
| | | GGCCTATCAACG (SEQ ID NO: 41) | |

### REFERENCES

1. Dedkova, L. M. and Hecht, S. M. (2019) Expanding the scope of protein synthesis using modified ribosomes. J. Am. Chem. Soc., 141, jacs.9b02109.
2. Hammerling, M. J., Krüger, A. and Jewett, M. C. (2019) Strategies for in vitro engineering of the translation machinery. Nucleic Acids Res., 10.1093/nar/gkz1011.
3. Chin, J. W. (2017) Expanding and reprogramming the genetic code. Nature, 550, 53-60.
4. Arranz-Gibert, P., Vanderschuren, K. and Isaacs, F. J. (2018) Next-generation genetic code expansion. Curr. Opin. Chem. Biol., 46, 203-211.
5. Tharp, J. M., Krahn, N., Varshney, U. and Söll, D. (2020) Hijacking translation initiation for synthetic biology. ChemBioChem, 21, 1387-1396.
6. Liu, C. C. and Schultz, P. G. (2010) Adding new chemistries to the genetic code. Annu. Rev. Biochem., 79, 413-44.
7. Rogers, J. M. and Suga, H. (2015) Discovering functional, non-proteinogenic amino acid containing, peptides using genetic code reprogramming. Org. Biomol. Chem., 13, 9353-9363.
8. Katoh, T. and Suga, H. (2018) Ribosomal incorporation of consecutive β-amino acids. J. Am. Chem. Soc., 140, 12159-12167.
9. Lee, J., Torres, R., Kim, D. S., Byrom, M., Ellington, A. D. and Jewett, M. C. (2020) Ribosomal incorporation of cyclic β-amino acids into peptides using in vitro translation. Chem. Commun., 56, 5597-5600.
10. Ohshiro, Y., Nakajima, E., Goto, Y., Fuse, S., Takahashi, T., Doi, T. and Suga, H. (2011) Ribosomal synthesis of backbone-macrocyclic peptides containing γ-amino acids. ChemBioChem, 12, 1183-1187.
11. Tsiamantas, C., Kwon, S., Rogers, J. M., Douat, C., Huc, I. and Suga, H. (2020) Ribosomal incorporation of aromatic oligoamides as peptide sidechain appendages. Angew. Chemie-Int. Ed., 59, 4860-4864.
12. Katoh, T. and Suga, H. (2020) Ribosomal elongation of cyclic γ-amino acids using a reprogrammed genetic code. J. Am. Chem. Soc., 142, 4965-4969.
13. Lee, J., Schwarz, K. J., Kim, D. S., Moore, J. S. and Jewett, M. C. (2020) Ribosome-mediated polymerization of long chain carbon and cyclic amino acids into peptides in vitro. Nat. Commun., 11, 4304.
14. Goto, Y., Murakami, H. and Suga, H. (2008) Initiating translation with D-amino acids. Rna, 14, 1390-1398.
15. Katoh, T., Tajima, K. and Suga, H. (2017) Consecutive elongation of D-amino acids in translation. Cell Chem. Biol., 24, 46-54.
16. Lee, J., Schwieter, K. E., Watkins, A. M., Kim, D. S., Yu, H., Schwarz, K. J., Lim, J., Coronado, J., Byrom, M., Anslyn, E. V., et al. (2019) Expanding the limits of the second genetic code with ribozymes. Nat. Commun., 10, 1-12.
17. Ad, O., Hoffman, K. S., Cairns, A. G., Featherston, A. L., Miller, S. J., Söll, D. and Schepartz, A. (2019) Translation of diverse aramid- and 1,3-dicarbonyl-peptides by wild type ribosomes in vitro. ACS Cent. Sci., 5, 1289-1294.
18. Kawakami, T., Ogawa, K., Hatta, T., Goshima, N. and Natsume, T. (2016) Directed evolution of a cyclized peptoid-peptide chimera against a cell-free expressed protein and proteomic profiling of the interacting proteins to create a protein-protein interaction inhibitor. ACS Chem. Biol., 11, 1569-1577.
19. Torikai, K. and Suga, H. (2014) Ribosomal synthesis of an amphotericin-B inspired macrocycle. J. Am. Chem. Soc., 136, 17359-17361.
20. Kawakami, T., Ishizawa, T. and Murakami, H. (2013) Extensive reprogramming of the genetic code for genetically encoded synthesis of highly N-alkylated polycyclic peptidomimetics. J. Am. Chem. Soc., 135, 12297-12304.
21. Goto, Y., Katoh, T. and Suga, H. (2011) Flexizymes for genetic code reprogramming. Nat. Protoc., 6, 779-790.
22. Goto, Y. and Suga, H. (2009) Translation initiation with initiator tRNA charged with exotic peptides. J. Am. Chem. Soc., 131, 5040-5041.
23. Oba, M. (2019) Cell-penetrating peptide foldamers: drug-delivery tools. ChemBioChem, 20, 2041-2045.
24. Rogers, J. M., Kwon, S., Dawson, S. J., Mandal, P. K., Suga, H. and Huc, I. (2018) Ribosomal synthesis and folding of peptide-helical aromatic foldamer hybrids. Nat. Chem., 10, 405-412.
25. Wang, K., Neumann, H., Peak-Chew, S. Y. and Chin, J. W. (2007) Evolved orthogonal ribosomes enhance the efficiency of synthetic genetic code expansion. Nat. Biotechnol., 25, 770-7.
26. Rackham, O. and Chin, J. W. (2005) A network of orthogonal ribosome-mRNA pairs. Nat. Chem. Biol., 1, 159-166.
27. Neumann, H., Wang, K., Davis, L., Garcia-Alai, M. and Chin, J. W. (2010) Encoding multiple unnatural amino acids via evolution of a quadruplet-decoding ribosome. Nature, 464, 441-4.
28. Maini, R., Chowdhury, S. R., Dedkova, L. M., Roy, B., Daskalova, S. M., Paul, R., Chen, S. and Hecht, S. M. (2015) Protein synthesis with ribosomes selected for the incorporation of β-amino acids. Biochemistry, 54, 3694-3706.
29. Dedkova, L. M., Fahmi, N. E., Golovine, S. Y. and Hecht, S. M. (2003) Enhanced D-amino acid incorporation into protein by modified ribosomes. J. Am. Chem. Soc., 125, 6616-6617.
30. Dedkova, L. M., Fahmi, N. E., Golovine, S. Y. and Hecht, S. M. (2006) Construction of modified ribosomes for incorporation of D-amino acids into proteins. Biochemistry, 45, 15541-15551.
31. Orelle, C., Carlson, E. D., Szal, T., Florin, T., Jewett, M. C. and Mankin, A. S. (2015) Protein synthesis by ribosomes with tethered subunits. Nature, 10.1038/nature14862.
32. Schmied, W. H., Tnimov, Z., Uttamapinant, C., Rae, C. D., Fried, S. D. and Chin, J. W. (2018) Controlling orthogonal ribosome subunit interactions enables evolution of new function. Nature, 564, 444-448.
33. Fried, S. D., Schmied, W. H., Uttamapinant, C. and Chin, J. W. (2015) Ribosome subunit stapling for orthogonal translation in E. coli. Angew. Chemie-Int. Ed., 54, 12791-12794.
34. Carlson, E. D., d'Aquino, A. E., Kim, D. S., Fulk, E. M., Hoang, K., Szal, T., Mankin, A. S. and Jewett, M. C. (2019) Engineered ribosomes with tethered subunits for expanding biological function. Nat. Commun., 10, 1-13.
35. Aleksashin, N. A., Leppik, M., Hockenberry, A. J., Klepacki, D., Vázquez-Laslop, N., Jewett, M. C., Remme, J. and Mankin, A. S. (2019) Assembly and functionality of the ribosome with tethered subunits. Nat. Commun., 10.
36. Cochella, L. and Green, R. (2004) Isolation of antibiotic resistance mutations in the rRNA by using an in vitro selection system. Proc. Natl. Acad. Sci. U.S.A, 101, 3786-91.
37. Hammerling, M. J., Fritz, B. R., Yoesep, D. J., Kim, D. S., Carlson, E. D. and Jewett, M. C. (2020) In vitro ribosome synthesis and evolution through ribosome display. Nat. Commun., 11, 1108.
38. Lu, T., Stroot, P. G. and Oerther, D. B. (2009) Reverse transcription of 16S rRNA to monitor ribosome-synthesizing bacterial populations in the environment. Appl. Environ. Microbiol., 75, 4589-4598.
39. Tsuji, H., Matsuda, K. and Nomoto, K. (2018) Counting the countless: Bacterial quantification by targeting rRNA molecules to explore the human gut microbiota in health and disease. Front. Microbiol., 9.
40. Matsuda, K., Tsuji, H., Asahara, T., Kado, Y. and Nomoto, K. (2007) Sensitive quantitative detection of commensal bacteria by rRNA-targeted reverse transcription-PCR. Appl. Environ. Microbiol., 73, 32-39.
41. Matsuda, K., Tsuji, H., Asahara, T., Takahashi, T., Kubota, H., Nagata, S., Yamashiro, Y. and Nomoto, K. (2012) Sensitive quantification of Clostridium difficile cells by reverse transcription-quantitative PCR targeting rRNA molecules. Appl. Environ. Microbiol., 78, 5111-5118.
42. Sakaguchi, S., Saito, M., Tsuji, H., Asahara, T., Takata, O., Fujimura, J., Nagata, S., Nomoto, K. and Shimizu, T. (2010) Bacterial rRNA-targeted reverse transcription-PCR used to identify pathogens responsible for fever with neutropenia. J. Clin. Microbiol., 48, 1624-1628.
43. Cox, C. J., Kempsell, K. E. and Gaston, J. S. H. (2003) Investigation of infectious agents associated with arthritis by reverse transcription PCR of bacterial rRNA. Arthritis Res. Ther., 5, 1-8.
44. Fujimori, M., Hisata, K., Nagata, S., Matsunaga, N., Komatsu, M., Shoji, H., Sato, H., Yamashiro, Y., Asahara, T., Nomoto, K., et al. (2010) Efficacy of bacterial ribosomal RNA-targeted reverse transcription-quantitative PCR for detecting neonatal sepsis: a case control study. BMC Pediatr., 10, 53.
45. Engstrand, L., Nguyen, A. M. H., Graham, D. Y. and El-Zaatari, F. A. K. (1992) Reverse transcription and polymerase chain reaction amplification of rRNA for detection of Helicobacter species. J. Clin. Microbiol., 30, 2295-2301.
46. Motorin, Y., Muller, S., Behm-Ansmant, I. and Branlant, C. (2007) Identification of modified residues in RNAs by reverse transcription-based methods. Methods Enzymol., 425, 21-53.
47. Cantara, W. A., Crain, P. F., Rozenski, J., McCloskey, J. A., Harris, K. A., Zhang, X., Vendeix, F. A. P., Fabris, D. and Agris, P. F. (2011) The RNA modification database, RNAMDB: 2011 update. Nucleic Acids Res., 39, 195-201.
48. Ellefson, J. W., Gollihar, J., Shroff, R., Shivram, H., Iyer, V. R. and Ellington, A. D. (2016) Synthetic evolutionary origin of a proofreading reverse transcriptase. Science (80-), 352, 1590-1593.
49. Sas-Chen, A. and Schwartz, S. (2019) Misincorporation signatures for detecting modifications in mRNA: Not as simple as it sounds. Methods, 156, 53-59.
50. Potapov, V., Fu, X., Dai, N., Corrêa, I. R., Tanner, N. A. and Ong, J. L. (2018) Base modifications affecting RNA polymerase and reverse transcriptase fidelity. Nucleic Acids Res., 46, 5753-5763.
51. Schwartz, S. and Motorin, Y. (2017) Next-generation sequencing technologies for detection of modified nucleotides in RNAs. RNA Biol., 14, 1124-1137.
52. Lee, J., Kladwang, W., Lee, M., Cantu, D., Azizyan, M., Kim, H., Limpaecher, A., Yoon, S., Treuille, A. and Das, R. (2014) RNA design rules from a massive open laboratory. Proc. Natl. Acad. Sci. U.S.A, 111, 2122-2127.
53. Masella, A. P., Bartram, A. K., Truszkowski, J. M., Brown, D. G. and Neufeld, J. D. (2012) PANDAseq: paired-end assembler for illumina sequences. BMC Bioinformatics, 13, 31.
54. Li, H. and Durbin, R. (2009) Fast and accurate short read alignment with Burrows-Wheeler transform. Bioinformatics, 25, 1754-1760.
55. Li, H., Handsaker, B., Wysoker, A., Fennell, T., Ruan, J., Homer, N., Marth, G., Abecasis, G. and Durbin, R. (2009) The Sequence Alignment/Map format and SAMtools. Bioinformatics, 25, 2078-2079.
56. Favero, F., Joshi, T., Marquard, A. M., Birkbak, N. J., Krzystanek, M., Li, Q., Szallasi, Z. and Eklund, A. C. (2015) Sequenza: Allele-specific copy number and mutation profiles from tumor sequencing data. Ann. Oncol., 26, 64-70.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for preparing a DNA molecule from an RNA template via reverse transcription, wherein the RNA template comprises one or more modified ribonucleosides, the method comprising reacting a reaction mixture comprising:
(i) a thermostable enzyme that comprises RNA-dependent DNA polymerase activity,
(ii) the RNA template,
(iii) one or more oligonucleotide primers that hybridize to the RNA template, and
(iv) reagents for performing reverse transcription of the RNA template.

2. The method of claim 1, wherein the one or more oligonucleotide primers hybridize to a region of the RNA template spanning the one or more modified ribonucleosides.

3. The method of claim 1, wherein the reaction mixture is reacted for at least about 14 minutes or longer.

4. The method of claim 1, wherein the reaction mixture is reacted for no more than about 6 minutes or less.

5. (canceled)

6. The method of claim 1, wherein the thermostable enzyme additionally comprises DNA-dependent DNA polymerase activity.

7. The method of claim 1, wherein the enzyme is RTX.

8. The method of claim 1, wherein the reaction mixture comprises an additional different enzyme that comprises DNA-dependent DNA polymerase activity.

9. The method of claim 1, wherein the reaction mixture further comprises:
(v) a forward primer and a reverse primer that hybridize to the DNA molecule, and
(vi) reagents for amplifying the prepared DNA molecule, and
the method further comprises amplifying the prepared DNA molecule via performing a polymerase chain reaction (PCR) amplification.

10. The method of claim 9, wherein the method comprises performing an elongation step in which reverse transcription of the RNA template occurs and an amplification step during which amplification of the prepared DNA occurs.

11. The method of claim 1, wherein the RNA template is at least about 200, 400, 600, 800, 1000, 1200, 1400, 1600, 1800, 2000, 2200, 2400, 2600, 2800, 3000 nucleotides in length or longer, and the method prepares a DNA molecule corresponding to the full-length RNA template.

12. The method of claim 1, wherein the RNA template comprises ribosomal RNA.

13-15. (canceled)

16. The method of claim 1, wherein the modified ribonucleoside is a naturally occurring modified ribonucleoside.

17. The method of claim 1, wherein the modified ribonucleoside comprises a methylated base.

18. The method of claim 1, wherein the modified ribonucleoside comprises a methylated ribose.

19. The method of claim 1, wherein the one or more modified ribonucleosides are selected from pseudouridine, N⁷-methylguanosine, N²-methylguanosine, N⁴-methylcytosine, N⁴,2′-O-methylcytosine, 5-methylcytosine, 5-hydroxymethylcytosine, N³-methyluridine, N⁶,N⁶-dimethyladenosine, N¹-methylguanosine, 5-methyluridine, N⁶-methyladenosine, N³-methylpseudouridine, 5-methyluridine, 2′-O-methylguanosine, dihydrouridine, 2′-O-methylcytosine, N²-methyladenosine, and 2′-O-methyluridine.

20-21. (canceled)

22. A method for identifying a modified ribonucleoside at a position in an RNA template, the method comprising:
(a) preparing a DNA molecule from the RNA template via reverse transcription by reacting a reaction mixture comprising:
(i) a thermostable enzyme that comprises RNA-dependent DNA polymerase activity,
(ii) the RNA template,
(iii) one or more oligonucleotide primers that hybridize to the RNA template, and
(iv) reagents for performing reverse transcription of the RNA template comprising deoxyribonucleotides which optionally are labeled; and
(b) identifying the incorporated deoxyribonucleotides in the DNA molecule,
(c) generating a mutation spectrum based on the incorporated deoxyribonucleotides at a position in the DNA molecule,
(d) comparing the generated mutation spectrum to a reference mutation spectrum which is characteristic of the modified ribonucleoside, and
(e) identifying the modified ribonucleoside in the RNA template at a position corresponding to the position in the DNA molecule.

23. The method of claim 22, wherein the modified ribonucleoside is selected from pseudouridine, N⁷-methylguanosine, N²-methylguanosine, N⁴-methylcytosine, N⁴,2′-O-methylcytosine, 5-methylcytosine, 5-hydroxymethylcytosine, N³-methyluridine, N⁶,N⁶-dimethyladenosine, N′-methylguanosine, 5-methyluridine, N⁶-methyladenosine, N³-methylpseudouridine, 5-methyluridine, 2′-O-methylguanosine, dihydrouridine, 2′-O-methylcytosine, N²-methyladenosine, and 2′-O-methyluridine.

24-25. (canceled)

26. A method for preparing a DNA molecule from an RNA template via reverse transcription, wherein the RNA template comprises one or more modified ribonucleosides, the method comprising reacting a reaction mixture comprising:
(i) a thermostable enzyme that comprises RNA-dependent DNA polymerase activity,
(ii) the RNA template,
(iii) one or more oligonucleotide primers that hybridize to the RNA template and comprise at least one bridging oligonucleotide primer that hybridizes to a region of the RNA template spanning the one or more modified ribonucleosides, and
(iv) reagents for performing reverse transcription of the RNA template.

27. The method of claim 26, wherein the modified ribonucleoside is selected from pseudouridine, N⁷-methylguanosine, N²-methylguanosine, N⁴-methylcytosine, N⁴,2′-O-methylcytosine, 5-methylcytosine, 5-hydroxymethylcytosine, N³-methyluridine, N⁶,N⁶-dimethyladenosine, N′-methylguanosine, 5-methyluridine, N⁶-methyladenosine, N³-methylpseudouridine, 5-methyluridine, 2′-O-methylguanosine, dihydrouridine, 2′-O-methylcytosine, N²-methyladenosine, and 2′-O-methyluridine.

28-30. (canceled)

31. The method of claim 26, wherein the enzyme is RTX.


SEQUENCE LISTING (43 sequences)

SEQ ID NO: 1 - DNA, 32 bp, Artificial Sequence
  Synthetic- 23S-rRNA-for
ggttaagcgactaagcgtacacggtggatgcc

SEQ ID NO: 2 - DNA, 60 bp, Artificial Sequence
  Synthetic- 23S_rRNA_784_for
ggaggaccgaaccgactaatgttgaaaaattagcggatgacttgtggctgggggtgaaag

SEQ ID NO: 3 - DNA, 60 bp, Artificial Sequence
  Synthetic- 23S_rRNA_708_rev
aagtcatccgctaatttttcaacattagtcggttcggtcctccagttagtgttacccaac

SEQ ID NO: 4 - DNA, 47 bp, Artificial Sequence
  Synthetic- 23S_rRNA_1867_for
acggtgtgacgcctgcccggtgccggaaggttaattgatggggttag

SEQ ID NO: 5 - DNA, 60 bp, Artificial Sequence
  Synthetic- 23S_rRNA_1800_rev
atcaattaaccttccggcaccgggcaggcgtcacaccgtatacgtccactttcgtgtttg

SEQ ID NO: 6 - DNA, 60 bp, Artificial Sequence
  Synthetic- 23s_rRNA_1954_for
cggtaaacggcggccgtaactataacggtcctaaggtagcgaaattccttgtcgggtaag

SEQ ID NO: 7 - DNA, 40 bp, Artificial Sequence
  Synthetic- 23S_rRNA_1881_rev
aggaccgttatagttacggccgccgtttaccggggcttcg

SEQ ID NO: 8 - DNA, 35 bp, Artificial Sequence
  Synthetic- 23S_rRNA_2282_for
ctggggcggtctcctcctaaagagtaacggaggag

SEQ ID NO: 9 - DNA, 60 bp, Artificial Sequence
  23S_rRNA_2218_rev
ccgttactctttaggaggagaccgccccagtcaaactacccaccagacactgtccgcaac

SEQ ID NO: 10 - DNA, 41 bp, Artificial Sequence
  Synthetic- 23s_rRNA_2535_for
gcacctcgatgtcggctcatcacatcctggggctgaagtag

SEQ ID NO: 11 - DNA, 60 bp, Artificial Sequence
  Synthetic- 23s_rRNA_2465_rev
ccaggatgtgatgagccgacatcgaggtgccaaacaccgccgtcgatatgaactcttggg

SEQ ID NO: 12 - DNA, 38 bp, Artificial Sequence
  Synthetic- 23s_rRNA_end_rev
aaggttaagcctcacggttcattagtaccggttagctc

SEQ ID NO: 13 - DNA, 42 bp, Artificial Sequence
  Synthetic- 16s_rRNA_42_for
aaattgaagagtttgatcatggctcagattgaacgctggcgg

SEQ ID NO: 14 - DNA, 28 bp, Artificial Sequence
  Synthetic- 16S_rRNA_543_for
tgccagcagccgcggtaatacggagggt

SEQ ID NO: 15 - DNA, 59 bp, Artificial Sequence
  Synthetic- 16S_rRNA_1002_for
ggagcatgtggtttaattcgatgcaacgcgaagaaccttacctggtcttgacatccacg

SEQ ID NO: 16 - DNA, 50 bp, Artificial Sequence
  Synthetic- 16S_rRNA_931_rev
ggttcttcgcgttgcatcgaattaaaccacatgctccaccgcttgtgcgg

SEQ ID NO: 17 - DNA, 37 bp, Artificial Sequence
  Synthetic- 16S_rRNA_1461_rev
cgacttcaccccagtcatgaatcacaaagtggtaagc

SEQ ID NO: 18 - DNA, 23 bp, Artificial Sequence
  Synthetic- 16S _rev
taaggaggtgatccaaccgcagg

SEQ ID NO: 19 - DNA, 47 bp, Artificial Sequence; identical to SEQ ID NO: 4
  Synthetic- product A, B, C, D, E, forward primer

SEQ ID NO: 20 - DNA, 60 bp, Artificial Sequence
  Synthetic- product A reverse primer
tcttgccgcgggtacactgcatcttcacagcgagttcaatttcactgagtctcgggtgga

SEQ ID NO: 21 - DNA, 60 bp, Artificial Sequence; identical to SEQ ID NO: 9
  Synthetic- Product B reverse primer

SEQ ID NO: 22 - DNA, 60 bp, Artificial Sequence; identical to SEQ ID NO: 11
  Synthetic- Product C reverse primer

SEQ ID NO: 23 - DNA, 20 bp, Artificial Sequence
  Synthetic- Product D reverse primer
cgacgttctaaacccagctc

SEQ ID NO: 24 - DNA, 23 bp, Artificial Sequence
  Synthetic- Product E reverse primer
ggcgttgtaaggttaagcctcac

SEQ ID NO: 25 - DNA, 60 bp, Artificial Sequence; identical to SEQ ID NO: 2
  Synthetic- Products F, G, H, I, J, forward primer

SEQ ID NO: 26 - DNA, 60 bp, Artificial Sequence; identical to SEQ ID NO: 20
  Synthetic- Product F reverse primer

SEQ ID NO: 27 - DNA, 60 bp, Artificial Sequence; identical to SEQ ID NO: 9
  Synthetic- Product G reverse primer

SEQ ID NO: 28 - DNA, 60 bp, Artificial Sequence; identical to SEQ ID NO: 11
  Synthetic- Product H reverse primer

SEQ ID NO: 29 - DNA, 20 bp, Artificial Sequence
  Synthetic- Product I reverse primer
cgacgttctaaacccagctc

SEQ ID NO: 30 - DNA, 23 bp, Artificial Sequence
  Synthetic- Product J reverse primer
ggcgttgtaaggttaagcctcac

SEQ ID NO: 31 - DNA, 60 bp, Artificial Sequence; identical to SEQ ID NO: 20
  Synthetic- Product K reverse primer

SEQ ID NO: 32 - DNA, 60 bp, Artificial Sequence; identical to SEQ ID NO: 9
  Synthetic- Product L reverse primer

SEQ ID NO: 33 - DNA, 60 bp, Artificial Sequence; identical to SEQ ID NO: 11
  Synthetic- Product M reverse primer

SEQ ID NO: 34 - DNA, 20 bp, Artificial Sequence
  Synthetic- Product N reverse primer
cgacgttctaaacccagctc

SEQ ID NO: 35 - DNA, 23 bp, Artificial Sequence
  Synthetic- Product O reverse primer
ccactccggtcctctcgtactag

SEQ ID NO: 36 - DNA, 23 bp, Artificial Sequence
  Synthetic- Product P-1 reverse primer
ggcgttgtaaggttaagcctcac

SEQ ID NO: 37 - DNA, 25 bp, Artificial Sequence
  Synthetic- Product P-2 reverse primer
aaggttaagcctcacggttcattag

SEQ ID NO: 38 - DNA, 31 bp, Artificial Sequence
  Synthetic- Product P-3 reverse primer
aaggttaagcctcacggttcattagtaccgg

SEQ ID NO: 39 - DNA, 38 bp, Artificial Sequence; identical to SEQ ID NO: 12
  Synthetic- Product P-4 reverse primer

SEQ ID NO: 40 - DNA, 37 bp, Artificial Sequence
  Synthetic- Product P-5 reverse primer
ggttcattagtaccggttagctcaacgcatcgctgcg

SEQ ID NO: 41 - DNA, 41 bp, Artificial Sequence
  Synthetic- Product P-6 reverse primer
gctcaacgcatcgctgcgcttacacacccggcctatcaacg

SEQ ID NO: 42 - DNA, 32 bp, Artificial Sequence; identical to SEQ ID NO: 1
  Synthetic- Products K, L, M, N, O, P-1-6 forward primer

SEQ ID NO: 43 - DNA, 60 bp, Artificial Sequence; identical to SEQ ID NO: 20
  Synthetic- 23S_rRNA_1995_rev